(19) United States
(12) Patent Application Publication
(10) Pub. No.: US 2024/0096469 A1
(43) Pub. Date: Mar. 21, 2024

(54) METHODS OF PREDICTING RESPONSES TO DISEASE TREATMENTS

(71) Applicant: Wisconsin Alumni Research Foundation, Madison, WI (US)

(72) Inventor: Shuang Zhao, Verona, WI (US)

(73) Assignee: Wisconsin Alumni Research Foundation, Madison, WI (US)

(21) Appl. No.: 18/370,296

(22) Filed: Sep. 19, 2023

Related U.S. Application Data
(60) Provisional application No. 63/408,316, filed on Sep. 20, 2022.

(51) Int. Cl. G16H 20/10; G16B 20/00

(52) U.S. Cl. CPC G16H 20/10; G16B 20/00

(57) ABSTRACT

Methods of generating linear regression predictor models capable of predicting responses of patients afflicted with diseases to treatments, methods of using the linear regression predictor models to predict the responses of the patients to the treatments, and methods of administering the treatments to the patients.

### CROSS-REFERENCE TO RELATED APPLICATIONS

Priority is hereby claimed to U.S. Provisional Application No. 63/408,316, filed Sep. 20, 2022, which is incorporated herein by reference.

### Electronic Tables

Tables 1A-1I have been submitted as ASCII text files via EFS-Web and are incorporated by reference.

The ASCII text file of Table 1A is named “Table_1A.txt,” was created on Sep. 19, 2022, and is 1,389,386 bytes in size.

The ASCII text file of Table 1B is named “Table_1B.txt,” was created on Sep. 19, 2022, and is 1,417,148 bytes in size.

The ASCII text file of Table 1C is named “Table_1C.txt,” was created on Sep. 19, 2022, and is 1,418,536 bytes in size.

The ASCII text file of Table 1D is named “Table_1D.txt,” was created on Sep. 19, 2022, and is 1,356,076 bytes in size.

The ASCII text file of Table 1E is named “Table_1E.txt,” was created on Sep. 19, 2022, and is 1,414,372 bytes in size.

The ASCII text file of Table 1F is named “Table_1F.txt,” was created on Sep. 19, 2022, and is 1,411,596 bytes in size.

The ASCII text file of Table 1G is named “Table_1G.txt,” was created on Sep. 19, 2022, and is 1,411,596 bytes in size.

The ASCII text file of Table 1H is named “Table_1H.txt,” was created on Sep. 19, 2022, and is 1,411,596 bytes in size.

The ASCII text file of Table 1I is named “Table _1I.txt,” was created on Sep. 19, 2022, and is 1,381,060 bytes in size.

### FIELD OF THE INVENTION

The invention is directed to methods of generating linear regression predictor models capable of predicting responses of patients afflicted with diseases to treatments, methods of using the linear regression predictor models to predict the responses of the patients to the treatments, and methods of administering the treatments to the patients.

### BACKGROUND

Many treatments demonstrate some efficacy in a minority of patients but lack sufficient clinical benefit in unselected populations to warrant FDA approval or clinical use. We are now in an era of molecular medicine where specific predictive biomarkers can be used to identify patients who will respond to specific treatments. To date, however, we lack a unified global approach for identifying the patients most likely to benefit from specific treatments, and there are only a handful of clinically used predictive biomarkers in the treatment of diseases such as cancer. Predictive, biomarker-based models that identify patients who will respond to specific treatments are needed.

### SUMMARY OF THE INVENTION

One aspect of the invention is directed to methods of predicting response of a patient afflicted with a disease to a treatment and, optionally, administering the treatment to the patient. In some versions, the methods comprise determining a gene expression level for each of one or more first genes in a patient sample comprising pathological patient cells, determining a mutation status for each of one or more second genes in the patient sample; and determining a treatment-response score from the one or more gene expression levels and the one or more mutation statuses in a linear regression predictor model. The linear regression predictor model in some versions, includes a predictor intercept, a predictor gene-expression coefficient for each of the one or more first genes, and a predictor mutation-status coefficient for each of the one or more second genes. The treatment-response score in some versions indicates a predicted response of the patient to the treatment.

Some versions comprise isolating the patient sample from the patient.

In some versions, determining the gene expression level for each of the one or more first genes comprises assaying the gene expression level of each of the one or more first genes in the patient sample. In some versions, determining the mutation status for each of the one or more second genes comprises assaying the mutation status of each of the one or more second genes in the patient sample.

In some versions, the patient is a cancer patient. In some versions, the treatment is a cancer treatment. In some versions, the patient sample comprises cancer cells.

In some versions, the mutation status indicates presence or absence of a coding mutation.

In some versions, the one or more first genes, the one or more second genes, the predictor intercept, the one or more predictor gene-expression coefficients, and the one or more predictor mutation-status coefficients are determined. In some versions, the process of determining these elements comprises identifying one or more disease-associated genes that are associated with the disease, determining treatment responses of training samples comprising pathological training cells subjected to the treatment, determining a gene expression level and a mutation status for each disease-associated gene in each training sample, and modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.

In some versions, the one or more first genes comprise all the disease-associated genes having a non-zero training gene-expression coefficient.

In some versions, the one or more second genes comprise all the disease-associated genes having a non-zero training mutation-status coefficient.

In some versions, the linear regression training model is a penalized linear regression model.

In some versions, the linear regression training model is an Elastic-Net regression model.

In some versions, determining the treatment responses of the training samples comprises assaying responses of the training samples to the treatment. In some versions, determining the gene expression level for each disease-associated gene in each training sample comprises assaying the gene expression level for each disease-associated gene in each training sample. In some versions, determining the mutation status for each disease-associated gene in each training sample comprises assaying the mutation status for each disease-associated gene in each training sample.

In some versions, the disease-associated genes are cancer-associated genes. In some versions, the training samples comprise cancer cells.

In some versions, the treatment is a treatment with a drug listed in Tables 1A-1I, In some versions, the one or more first genes comprise any one or more genes listed in Tables 1A-1I that have a non-zero gene-expression coefficient for the drug, the one or more second genes comprise any one or more genes listed in Tables 1A-1I that have a non-zero mutation-status coefficient for the drug, the predictor intercept is an approximate of the intercept listed in Tables 1A-1I for the drug, each predictor gene-expression coefficient is an approximate of the gene-expression coefficient for one of the one or more first genes listed in Tables 1A-1I for the drug, and each predictor mutation-status coefficient is an approximate of the mutation-status coefficient for one of the one or more second genes listed in Tables 1A-1I for the drug.

In some versions, the one or more first genes comprise all the genes listed in Tables 1A-1I that have a non-zero coefficient for the drug.

In some versions, the one or more second genes comprise all the genes listed in Tables 1A-1I having a non-zero coefficient for the drug.

In some versions, the determining the treatment-response score comprises determining a treatment-response score for more than one drug listed in Tables 1A-1I using a different linear regression predictor model for each of the more than one drug.

Some versions further comprise administering the treatment to the patient.

Some versions further comprise administering the treatment to the patient if the treatment-response score is within a therapeutic range.

In some versions, the administering ameliorates the disease.

Another aspect of the invention is directed to methods of generating a linear regression predictor model capable of predicting response of a patient afflicted with a disease to a treatment. The linear regression predictor model so developed can comprise one or more first genes, one or more second genes, a predictor intercept, one or more predictor gene-expression coefficients, and one or more predictor mutation-status coefficients. In some versions, the methods comprise identifying one or more disease-associated genes that are associated with the disease, determining treatment responses of training samples comprising pathological training cells subjected to the treatment, determining a gene expression level and a mutation status for each disease-associated gene in each training sample, and modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.

In some versions, the one or more first genes comprise all the disease-associated genes having a non-zero training gene-expression coefficient.

In some versions, the one or more second genes comprise all the disease-associated genes having a non-zero training mutation-status coefficient.

In some versions, the linear regression training model is a penalized linear regression model.

In some versions, the linear regression training model is an Elastic-Net regression model.

In some versions, determining the treatment responses of the training samples comprises assaying responses of the training samples to the treatment. In some versions, determining the gene expression level for each disease-associated gene in each training sample comprises assaying the gene expression level for each disease-associated gene in each training sample. In some versions, determining the mutation status for each disease-associated gene in each training sample comprises assaying the mutation status for each disease-associated gene in each training sample.

In some versions, the disease-associated genes are cancer-associated genes. In some versions, the training samples comprise cancer cells.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

FIG. 1. TARGETS Workflow. Flowchart depicting TARGETS model training on DNA and RNA sequencing data from the Genomics of Drug Sensitivity in Cancer (GDSC) database to predict drug response. Model training was performed utilizing Elastic Net regression. The drug models were locked and subsequently validated on three independent cohorts: the Cancer Cell Line Encyclopedia (CCLE), The Cancer Genome Atlas (TCGA) pan-cancer database, and the West Coast Dream Team (WCDT) metastatic prostate cancer database.

FIGS. 2A-2C. TARGETS Scores in TCGA patients. TARGETS predictions for all drugs (rows) across all samples in the TCGA (columns) are shown in heatmap form. Hierarchical clustering was performed on both rows and columns. A lower TARGETS score indicates predicted sensitivity. Cancer site and type is identified using the TCGA standardized study abbreviations. FIGS. 2A-2C depict a single heatmap split across each consecutive subfigure (FIGS. 2A, 2B, and 2C), with a slight overlap of data being reproduced in each consecutive subfigure pair. Hierarchical clustering lines indicated with *, #, **, and # are continued across consecutive subfigure pairs.

FIGS. 3A-3C. TARGETS concordance with FDA-approved and clinically used biomarker indications. Boxplots comparing predicted TARGETS drug sensitivity among patients with or without a specific biomarker. Distributions were compared using an unpaired two-sample t-test, with p-value reported. A lower TARGETS score indicates increased predicted sensitivity. Cancer site and type for each plot is identified using the TCGA standardized study abbreviations. Boxplot center line=median; box limits=upper and lower quartiles; whiskers=1.5×interquartile range.

FIGS. 4A and 4B. Predicting response to ARSIs in mCRPC. (FIG. 4A) Patients receiving ARSIs who had a 51-100% response in PSA (right boxplots in each boxplot pair of ARSI treatment conditions (no or yes)) were predicted to be more sensitive by TARGETS than those with a 0-50% response (right boxplots in each boxplot pair of ARSI treatment conditions (no or yes)). There was no difference in predicted sensitivity in those not treated. Boxplot center line=median; box limits=upper and lower quartiles; whiskers=1.5×interquartile range. (FIG. 4B) Interaction plot showing the probability of PSA 51-100% response as a function of TARGETS score in the patients treated with an ARSI vs. other treatments (Other Tx). A lower TARGETS score indicates predicted sensitivity.

FIGS. 5A-5C. Novel mutations predicted to confer drug sensitivity. FIG. 5A. List of top 1% mutations predicted to confer sensitivity to a specific agent across tumor types. Linear model p-values all<0.0001. The rank is based on the normalized weighting of the model with each model. FIG. 5B. Differences in TARGETS scores across 32 cancer types for Linsitinib. FIG. 5C. Differences in TARGETS scores across 32 cancer types for Elesclomol. A lower TARGETS score indicates predicted sensitivity. Boxplot center line=median; box limits=upper and lower quartiles; whiskers=1.5×interquartile range. In FIGS. 5B and 5C, the left boxplot in each pair of boxplots for each cancer type indicates an IDH1 status of “no,” and the right boxbplot in each pair of boxplots for each cancer type indicates an IDH1 status of “yes,” noting that only an IDH1 status of “no” is shown for cancer types UCS, TGCT, MESO, OV, ACC, DLBC, UVM, KIRP, THCA, and KICH in FIG. 5B and PCPG, UVM, ACC, and KICH in FIG. 5C.

### DETAILED DESCRIPTION OF THE INVENTION

One aspect of the invention is directed to linear regression models capable of predicting responses of patient afflicted with diseases to treatments. Such models are referred to herein as “linear regression predictor models.”

The linear regression predictor models of the invention comprise standard linear models that are specific to particular treatments of particular diseases. The linear regression predictor models include an intercept, referred to herein as a “predictor intercept”; a coefficient for the gene expression level of each of one or more first genes, referred to herein as “predictor gene-expression coefficients”; and a coefficient for the mutation status of each of one or more second genes, referred to herein as “predictor mutation-status coefficients.”

The diseases specific to the linear regression predictor models of the invention can be any disease. “Disease” as used herein encompasses any type of pathological state of an organism. Exemplary diseases include infectious diseases, deficiency diseases, hereditary diseases (including both genetic diseases and non-genetic hereditary diseases), physiological diseases, cancer, immune disease, substance dependence, cardiovascular disease, endocrine diseases, and metabolic diseases, among others. Exemplary types of cancer include colorectal cancer, pancreatic cancer, hepatocellular carcinoma, gastric cancer, glioma, thyroid cancer, acute myeloid leukemia, chronic myeloid leukemia, basal cell carcinoma, melanoma, renal cell carcinoma, bladder cancer, prostate cancer, endometrial cancer, breast cancer, small cell lung cancer, and non-small cell lung cancer, among others. Exemplary types of immune disease include asthma, systemic lupus erythematosus, rheumatoid arthritis, autoimmune thyroid disease, inflammatory bowel disease, allograft rejection, graft-versus-host disease, and primary immunodeficiency. Exemplary types of neurodegenerative disease include Alzheimer's disease, Parkinson's disease, amyotrophic lateral sclerosis, Huntington's disease, spinocerebellar ataxia, prion disease, and neurodegeneration diseases, among others. Exemplary types of substance dependence include cocaine addiction, amphetamine addiction, morphine addiction, nicotine addiction, and alcoholism, among others. Exemplary types of cardiovascular disease include hyperlipidemia, atherosclerosis, hypertrophic cardiomyopathy, arrhythmogenic right ventricular cardiomyopathy, dilated cardiomyopathy, diabetic cardiomyopathy, and viral myocarditis, among others. Exemplary types of endocrine and/or metabolic diseases include type II diabetes mellitus, type I diabetes mellitus, maturity onset diabetes of the young, alcoholic liver disease, non-alcoholic fatty liver disease, insulin resistance, and Cushing syndrome, among others.

The particular treatments specific to the linear regression predictor models of the invention can be any type of disease treatment. “Treatment” as used herein refers to any intervention to or with a patient, such as physical and/or psychological interventions, intended to ameliorate a disease. Exemplary treatments include administration of single drugs, administration of combinations of drugs, surgeries, radiation therapies, biological therapies, immunological therapies, hormonal therapies, medical procedures, psychotherapies, and any combination of the foregoing. See, e.g., US 2018/0291459 and US 2021/0317535, for various exemplary cancer therapies.

The first genes in the linear regression predictor models of the invention comprise genes whose expression levels are predictive in determining the responsiveness to a treatment. The second genes in the linear regression predictor models of the invention comprise genes whose mutation statuses are predictive in determining the responsiveness to a treatment. The first genes and second genes can be completely overlapping sets of genes, partially overlapping sets of genes, or completely different (non-overlapping) sets of genes. Exemplary methods of determining suitable first and second genes are described elsewhere herein.

The gene expression levels are preferably represented in the linear regression predictor models of the invention as normalized values representing relative levels of expression of a population of genes across multiple different samples, as applicable. Exemplary methods of gene-expression normalization are provided below.

“Mutation status” as used herein refers to the property of either having or not having a particular type of mutation in a given gene. The property of having a mutation can be represented in the exemplary linear regression predictor models as “1,” and the property of not having a mutation can be represented as “0.” The mutations are determined with respect to a reference genome. The reference genome is preferably the genome from a subject or cell that is not determinately characterized by a particular disease state. A number of reference genomes are publicly available. An exemplary reference genome is GRCh38/hg38 (Schneider V A, Graves-Lindsay T, Howe K, Bouk N, Chen H C, Kitts PA, et al. (May 2017). “Evaluation of GRCh38 and de novo haploid genome assemblies demonstrates the enduring quality of the reference assembly”. Genome Research. 27 (5): 849-864). The type of mutation represented by the mutation status in the model can comprise or consist of any type of mutation in a nucleic acid sequence, including substitutions, deletions, and/or insertions. The mutations can comprise or consist of non-coding mutations, coding mutations, or a combination thereof. In exemplary versions of the invention, the mutations represented by the mutation status in the models consist of coding mutations.

The linear regression predictor models of the invention can accordingly be in the form:

Treatment-Response Score=(E₁*+(E₂*X₂)+ . . . +(Eₙ+Xₙ)+(M₁*+Y₁)+(M₂*Y₂)++(Mₙ*Yₙ)+B

### Where:

E₁, E₂, . . . Eₙ are the gene-expression coefficients for genes 1, 2, . . . n for a given treatment.
X₁, X₂, . . . Xₙ are the normalized gene expression values for genes 1, 2, . . . n.
M₁, M₂, . . . Mₙ are the mutation-status coefficients for genes 1, 2, . . . n.
Y₁, Y₂, . . . Yₙ are the mutation statuses of genes 1, 2, . . . n (0 if not mutated, 1 if mutated).
B is the intercept.

The linear regression predictor models of the invention can be generated for a particular disease and disease treatment by a variety of methods. A preferred method can comprise a step of identifying genes that are associated with the disease. Genes associated with a given disease are referred to herein as “disease-associated genes.” Disease-associated genes are genes involved in the etiology of a particular disease. In some versions, the disease-associated genes comprise or consist of genes in which one or more mutations in those genes are associated with the disease, such as in a statistically significant manner. A number of databases are available that catalog disease-associated genes, including gene mutations associated with various diseases. The COSMIC (the Catalogue of Somatic Mutations in Cancer) database (cancer.sanger.ac.uk/cosmic), for example, is a database of somatically acquired mutations found in human cancer (Tate J G, et al. COSMIC: the catalogue of somatic mutations in cancer. Nucleic Acids Res. 2019;47:D941-D947). The DisGeNET (disgenet.org) database is a platform containing one of the largest publicly available collections of genes and variants associated with human diseases (Piñero J, Saüch J, Sanz F, Furlong L I. The DisGeNET cytoscape app: Exploring and visualizing disease genomics data. Comput Struct Biotechnol J. 2021 May 11; 19:2960-2967) (Piñero J, Ramírez-Anguita J M, Saüch-Pitarch J, Ronzano F, Centeno E, Sanz F, Furlong L I. The DisGeNET knowledge platform for disease genomics: 2019 update. Nucleic Acids Res. 2020 Jan. 8; 48(D1):D845-D855) (Piñero J, Bravo À, Queralt-Rosinach N, Gutíerrez-Sacristán A, Deu-Pons J, Centeno E, Garcia-Garcia J, Sanz F, Furlong L I. DisGeNET: a comprehensive platform integrating information on human disease-associated genes and variants. Nucleic Acids Res. 2017 Jan. 4; 45(D1):D833-D839) (Piñero J, Queralt-Rosinach N, Bravo À, Deu-Pons J, Bauer-Mehren A, Baron M, Sanz F, Furlong L I. DisGeNET: a discovery platform for the dynamical exploration of human diseases and their genes. Database (Oxford). 2015 Apr. 15; 2015:bav028). A large number of other databases are available (Babbi G, Martelli P L, Profiti G, Bovo S, Savojardo C, Casadio R. eDGAR: a database of Disease-Gene Associations with annotated Relationships among genes. BMC Genomics. 2017 Aug. 11; 18(Suppl 5):554) (Grissa D, Junge A, Oprea T I, Jensen L J. Diseases 2.0: a weekly updated database of disease-gene associations from text mining and data integration. Database (Oxford). 2022 Mar. 28; 2022:baac019).

The methods of generating the linear regression predictor models can further include a step of determining treatment responses of training samples comprising pathological training cells subjected to the treatment.

“Pathological cell” and variants thereof (“pathological training cell” or “pathological patient cell”), refers to a cell that is associated with, results from, causes, or contributes to a particular disease. In some versions, the pathological cell can have at least one structural or functional abnormality with regard to a healthy cell. The pathological cells can be physiological pathological cells or model pathological cells. Physiological pathological cells are pathological cells within or isolated directly from a patient (e.g., cancer cells within the body of the patient, primary cells isolated directly from the body of the patient, etc.). Model pathological cells are cells that mimic or model physiological pathological cells (e.g., cancer cell lines, ex vivo immortalized cells, etc.). Model pathological cells can be derived from primary cells or generated de novo. Exemplary pathological cells include cancer cells. Exemplary pathological cells of various immunological diseases include immune cells. Pathological cells of other diseases are well known in the art.

“Treatment response” refers to any effect of a treatment on a cell. The effect can be a deleterious effect, i.e., an effect that exacerbates any aspect of the disease, an ameliorative effect, i.e., an effect that ameliorates any aspect of the disease, or no effect. For a disease such as cancer, an exemplary treatment response can be the rate of cell division or cell survival. It is understood that any treatment responses incorporated in the linear regression predictor models are represented by quantitative values.

The step of determining the treatment responses of the training samples can be performed by any of a number of methods. In some versions, determining the treatment responses comprises assaying responses of the training samples to the treatment. “Assaying” as used herein refers to physically conducting experimental procedures on a material, thereby resulting in a change of physical state or structure. In the case of assaying responses of the training samples to the treatment, the assaying can comprise physically subjecting the training samples to the treatment and measuring the treatment responses on the samples. Such assaying can be performed in vivo, in vitro, or ex vivo. In some versions, determining the treatment responses comprises obtaining previously assayed responses, such as from a database containing such data.

The methods of generating the linear regression predictor models can further include a step of determining gene expression levels for each disease-associated gene in each training sample. This step can be performed by any of a number of methods. In some versions, determining the gene expression levels comprises assaying for the gene expression levels. Assaying the gene expression levels can comprise measuring the mRNA levels (e.g., measuring the number of mRNA copies) of different mRNA species present in the cells. Methods for assaying gene expression levels are well known in the art, and include RNA-seq, among other methods. See Stark et al. 2019 (Stark R, Grzelak M, Hadfield J. RNA sequencing: the teenage years. Nat Rev Genet. 2019 November; 20(11):631-656). In some versions, determining the gene expression levels comprises obtaining previously assayed gene expression levels, such as from a database containing such data. The gene expression levels incorporated in the linear regression models described herein (whether training or predictor models) are preferably values normalized from the raw, absolute values obtained with the assaying. An exemplary method of normalizing the gene expression levels for incorporating in the linear regression models is as follows. For a given set of gene expression values for a gene X in a cohort of samples, for example: Gene X in sample 1=1; Gene X in sample 2=3; Gene X in sample 3=7. A first step can include log 2 transforming the raw gene expression levels after adding 1: Log 2 Gene X in sample 1=log 2(1+1)=1; Log 2 Gene X in sample 2=log 2(3+1)=2; Log 2 Gene X in sample 3=log 2(7+1)=3. In a second step, the median of values of all the genes evaluated across all the samples is then divided from each value to complete the normalization. For example, if the median of values of all the genes evaluated across all the samples (not just Gene X, but also Gene Y, Z, etc.) is 1.5, then the median scaled Log 2 Gene X in sample 1=1/1.5=0.6667; the median scaled Log 2 Gene X in sample 2=2/1.5=1.3333; and the median scaled Log 2 Gene X in sample 3=3/1.5=2. In some versions, determining the gene expression levels of the disease-associated genes in the training samples excludes determining the gene expression levels of all of the genes in the training samples. In some versions, the gene expression levels of less than 75%, less than 50%, less than 25%, less than 15%, less than 10%, or less than 5% of the total number of genes in the training samples are determined.

The methods of generating the linear regression predictor models can further include a step of determining mutation statuses for each disease-associated gene in each training sample. This step can be performed by any of a number of methods. In some versions, determining the mutation statuses comprises assaying for the mutation statuses. Assaying the mutation statues can comprise sequencing the disease-associated genes and determining mutations therefrom. Sequencing the genes can comprise sequencing the gene, sequencing mRNA from the gene and deducing the gene sequence from the mRNA sequence, or other methods. In some versions, determining the mutation statuses comprises obtaining previously assayed mutation statuses, such as from a database containing such data. Positive mutation statuses in exemplary linear regression training models can be represented as “1,” and negative mutation statuses in the exemplary linear regression training models can be represented as “0.” In the exemplary linear regression training models, only coding mutations are assigned positive mutation statuses. In some versions, determining the mutation statuses of the disease-associated genes in the training samples excludes determining the mutation statuses of all of the genes in the training samples. In some versions, the mutation statuses of less than 75%, less than 50%, less than 25%, less than 15%, less than 10%, or less than 5% of the total number of genes in the training samples are determined.

As used herein, a given “sample” of cells, such as a training sample or a patient sample, refers to one or more physical sets of cells (e.g., pathological patient cells or pathological training cells) that are within, isolated from, or derived from a single source. The single source can be a particular patient, a particular patient tissue, a particular patient bodily fluid, a particular cell line, a particular primary cell bank, a particular primary tissue bank, etc. The various physical sets of cells of a given sample are understood to be representative of each other at least with respect to treatment responses, gene expression levels, and mutation statuses. Thus, a given sample of pathological training cells in which the treatment response, gene expression levels, and mutation statuses are determined can be different physical sets of cells, so long as the cells in each physical set are representative of each other with regard to these characteristics. Two or more physical sets of cells are be considered to constitute different samples if they are within, isolated from, or derived from different sources and/or have a difference in a treatment response to a particular drug, a gene expression level of a particular gene, and/or a mutation status of a particular gene.

In some versions, the methods of generating the linear regression predictor models can include determining treatment responses, gene expression levels, and mutation statuses of different training samples. In some versions, the different samples of pathological training cells may have at least one difference in the gene expression levels of a particular gene, at least one difference in the mutation status of a particular gene, and/or at least difference in specific mutations in a particular gene.

Once the treatment responses, the gene expression levels, and the mutation statuses are determined, these values can be modeled in a linear regression model, referred to herein as a “linear regression training model.” In preferred versions, the linear regression training model is a penalized linear regression model. Penalized linear regression models are models that are penalized for having too many variables in the model, for example, by adding a constraint in the equation (James, Gareth, Daniela Witten, Trevor Hastie, and Robert Tibshirani. 2014. An Introduction to Statistical Learning: With Applications in R. Springer Publishing Company, Incorporated). Penalized linear regression models are also known as shrinkage or regularization methods. Exemplary penalized linear regression models include Ridge regression, LASSO (least absolute shrinkage and selection operator) regression, and Elastic Net regression.

Modeling treatment responses, the gene expression levels, and the mutation statuses in the linear regression training model can thereby generate determine an intercept, referred to herein as a “training intercept”; a coefficient for the gene expression of each disease-associated gene, referred to herein as a “a training gene-expression coefficient”; and a coefficient for the mutation status of each disease-associated gene, referred to herein as a “training mutation-status coefficient.” Some or all of these values can be used to define the elements in the linear regression predictor models of the invention. For example, predictor intercept can be defined as the training intercept. The one or more first genes can be defined as any one or more of the disease-associated genes having a non-zero training gene-expression coefficient. The one or more second genes can be defined as any one or more of the disease-associated genes having a non-zero training mutation-status coefficient. The one or more predictor gene-expression coefficients can be defined as the training gene-expression coefficients of the disease-associated genes constituting the one or more first genes. The one or more predictor mutation-status coefficients can be defined as the training mutation-status coefficients of the disease-associated genes constituting the one or more second genes. Disease-associated genes having training gene-expression coefficients or mutation-status coefficients with a value of zero can be excluded from the first genes or the second genes, respectively, in any linear regression predictor models, as gene-expression values and the mutation statuses of such genes would not affect the treatment-response score generated from such models.

In some versions, the one or more first genes comprise at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least of 100 of the disease-associated genes having a non-zero training gene-expression coefficient. In some versions, the one or more first genes comprise all the disease-associated genes having a non-zero training gene-expression coefficient. In some versions, the one or more second genes comprise at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least of 100 of the disease-associated genes having a non-zero training mutation-status coefficient. In some versions, the one or more second genes comprise all the disease-associated genes having a non-zero training mutation-status coefficient.

The linear regression predictor models of the invention can be used in methods of predicting the response of a patient afflicted with a disease to a treatment. “Afflicted with a disease” as used herein refers to the state of having a disease. “Patient” as used herein refers to any subject, such as an animal subject, a mammalian subject, or a human subject.

The methods of predicting the response of a treatment can include a step of determining a gene expression level for each of the one or more of the first genes in a patient sample. The patient sample can comprise any sample within or derived from a patient that comprises pathological cells of the patient. The patient sample can be a tissue or fluid sample. The patient sample can be derived from any organ, tissue, or biological fluid. A patient sample can comprise, for example, a bodily fluid or a solid tissue sample. An example of a solid tissue sample is a tumor sample, e.g., from a solid tumor biopsy. Bodily fluids include, for example, blood serum, plasma, tumor cells, saliva, urine, lymphatic fluid, prostatic fluid, seminal fluid, milk, sputum, stool, tears, and derivatives of these. In some versions, the patient sample is isolated from the patient's body.

Determining a gene expression level for each of the one or more of the first genes can be performed by any of a number of methods. In some versions, determining the gene expression levels comprise assaying for the gene expression levels. Assaying the gene expression levels can comprise measuring the mRNA levels (e.g., measuring the number of mRNA copies) of different mRNA species present in the cells. In some versions, determining the gene expression levels comprise obtaining previously assayed gene expression levels, such as from a database containing such data. As outlined above, the gene expression levels incorporated in the linear regression predictor models described herein are preferably values normalized from the raw, absolute values obtained with the assaying. An exemplary method for normalizing the gene expression levels from a patient sample for incorporating in the linear regression predictor models could comprise collecting raw gene expression levels from a group of N control samples that are similarly processed as the patient sample, where N>1. The median of values of all the genes evaluated across all the control samples can then be divided from each value (from both control and patient samples) to complete the normalization. In some versions, determining the gene expression levels of for each of the one or more of the first genes in the patient sample excludes determining the gene expression levels of all of the genes in the patient sample. In some versions, the gene expression levels of less than 75%, less than 50%, less than 25%, less than 15%, less than 10%, or less than 5% of the total number of genes in the patient sample are determined.

The methods of predicting the response of a treatment can further include a step of determining a mutation status for each of one or more second genes in the patient sample. This step can be performed by any of a number of methods. In some versions, determining the mutation statuses comprises assaying for the mutation statuses. Assaying the mutation statues can comprise sequencing the disease-associated genes and determining mutations therefrom. Sequencing the genes can comprise any process described elsewhere herein. In some versions, determining the mutation statuses comprises obtaining previously assayed mutation statuses, such as from a database containing such data. As outlined above, positive mutation statuses in the exemplary linear regression predictor models are represented as “1,” and negative mutation statuses in the exemplary linear regression models are represented as “0.” In some versions of the invention, only coding mutations are assigned positive mutation statuses. In some versions of the invention, determining the mutation statuses of the disease-associated genes in the patient sample excludes determining the mutation statuses of all of the genes in the patient sample. In some versions, the mutation statuses of less than 75%, less than 50%, less than 25%, less than 15%, less than 10%, or less than 5% of the total number of genes in the patient sample are determined.

The methods of predicting the response of a treatment can further include a step of determining a treatment-response score from the one or more gene expression levels and the one or more mutation statuses in one or more of the linear regression predictor models of the invention. The treatment-response score can be defined as the sum of the predictor intercept, the product(s) of each first gene's gene expression level to its corresponding predictor gene-expression coefficient, and the product(s) of each second gene's mutation status to its corresponding predictor mutation-status coefficient. The treatment-response score indicates a predicted response of the patient to the treatment. An exemplary treatment-response score described in the following examples is referred to as a “TARGETS score.”

In some versions of the invention, the patient is a cancer patient, the modeled treatment is a cancer treatment, and the pathological cells from the patient are cancer cells from the patient.

In some versions, the modeled cancer treatment is a treatment with a drug, such as by administering the drug to the patient. In some versions, the methods of predicting the response of a treatment can include predicting the response of a treatment with any one or more of the following drugs, in any combination: Erlotinib, Rapamycin, Sunitinib, PHA-665752, MG-132, Paclitaxel, Cyclopamine, AZ628, Sorafenib, Tozasertib, Imatinib, NVP-TAE684, Crizotinib, Saracatinib, S-Trityl-L-cysteine, Z-LLNle-CHO, Dasatinib, GNF-2, CGP-60474, CGP-082996, A-770041, WH-4-023, WZ-1-84, BI-2536, BMS-536924, BMS-509744, CMK, Pyrimethamine, JW-7-52-1, A-443654, GW843682X, Entinostat, Parthenolide, GSK319347A, TGX221, Bortezomib, XMD8-85, Seliciclib, Salubrinal, Lapatinib, GSK269962A, Doxorubicin, Etoposide, Gemcitabine, Mitomycin-C, Vinorelbine, NSC-87877, Bicalutamide, QS11, CP466722, Midostaurin, CHIR-99021, Ponatinib, AZD6482, JNK-9L, PF-562271, HG6-64-1, JQ1, JQ12, DMOG, FTI-277, OSU-03012, Shikonin, AKT inhibitor, VIII, Embelin, FH535, PAC-1 IPA-3, GSK650394, BAY-61-3606, 5-Fluorouracil, Thapsigargin, Obatoclax, Mesylate, BMS-754807, Linsitinib, Bexarotene, Bleomycin, LFM-A13, GW-2580, Luminespib, Phenformin, Bryostatin 1, Pazopanib, Dacinostat, Epothilone B, GSK1904529A, BMS-345541, Tipifarnib, Avagacestat, Ruxolitinib, AS601245, Ispinesib, Mesylate, TL-2-105, AT-7519, TAK-715, BX-912, ZSTK474, AS605240, Genentech, Cpd 10, GSK1070916, Enzastaurin, GSK429286A, FMK, QL-XII-47, IC-87114, Idelalisib, UNC0638, Cabozantinib, WZ3105, XMD14-99, Quizartinib, CP724714, JW-7-24-1, NPK76-II-72-1, STF-62247, NG-25, TL-1-85, VX-11e, FR-180204, ACY-1215, Tubastatin, A Zibotentan, Sepantronium bromide, NSC-207895, VNLG/124, AR-42, CUDC-101, Belinostat, I-BET-762, CAY10603, Linifanib, BIX02189, Alectinib, Pelitinib, Omipalisib, JNJ38877605, SU11274, KIN001-236, KIN001-244, WHI-P97, KIN001-042, KIN001-260, KIN001-266, Masitinib, Amuvatinib, MPS-1-IN-1, NVP-BHG712 OSI-930, OSI-027, CX-5461, PHA-793887, PI-103, PIK-93, SB52334, TPCA-1, Fedratinib, Foretinib, Y-39983, YM201636, Tivozanib, WYE-125132, GSK690693, SNX-2112, QL-XI-92, XMD13-2, QL-X-138, XMD15-27, T0901317, Selisistat, Tenovin-6, THZ-2-49, KIN001-270, THZ-2-102-1, AT7867, CI-1033, PF-00299804, TWS119, Torin 2, Pilaralisib, GSK1059615, Voxtalisib, Brivanib, BMS-540215, BIBF-1120, AST-1306, Apitolisib, LIMK1, inhibitor, BMS4, kb NB 142-70, Sphingosine Kinase 1 Inhibitor II, eEF2K Inhibitor A-484954, MetAP2 Inhibitor A832234, Venotoclax, CPI-613, CAY10566, Ara-G, Pemetrexed, Alisertib, Flavopiridol, C-75, CAP-232 (CAP-232, TT-232, TLN-232), Trichostatin A, Panobinostat, LCL161, IMD-0354, MIM1, ETP-45835, CD532 NSC319726, ARRY-520, SB505124, A-83-01, LDN-193189, FTY-720, BAM7 AGI-6780, Kobe2602, LGK974, Wnt-059, RU-SKI 43, AICA Ribonucleotide, Vinblastine, Cisplatin, Cytarabine, Docetaxel, Methotrexate, Tretinoin, Gefitinib, Navitoclax, Vorinostat, Nilotinib, Refametinib, CI-1040, Temsirolimus, Olaparib, Veliparib, Bosutinib, Lenalidomide, Axitinib, AZD7762, GW441756, Lestaurtinib, SB216763, Tanespimycin, VX-702, Motesanib, KU-55933, Elesclomol, Afatinib, Vismodegib, PLX-4720, BX795, NU7441, SL0101, Doramapimod, JNK Inhibitor VIII, Weel Inhibitor, Nutlin-3a (−), Mirin, PD173074, ZM447439, RO-3306, MK-2206, Palbociclib, Dactolisib, Pictilisib, AZD8055, PD0325901, SB590885, Selumetinib, CCT007093, EHT-1864, Cetuximab, PF-4708671, Serdemetan, AZD4547, Capivasertib, HG-5-113-01, HG-5-88-01, TW 37, XMD11-85h, ZG-10, XMD8-92, QL-VIII-58, CCT-018159, Rucaparib, AZ20, KU-60019, Tamoxifen, QL-XII-61, PFI-1, IOX2, YK-4-279, (5Z)-7-Oxozeaenol, Piperlongumine, Daporinad, Talazoparib, rTRAIL, UNC1215, UNC0642, SGC0946, ICL1100013, XAV939, Trametinib, Dabrafenib, Temozolomide, Bleomycin (50 uM), AZD3514, Bleomycin (10 uM), AZD6738, AZD5438, AZD6094, Dyrklb_0191, AZD4877, EphB4_9721, Fulvestrant, AZD8931, FEN1_3940, FGFR_0939, FGFR_3831, BPTES, AZD7969, AZD5582, IAP_5620, IAP_7638, IGFR_3801, AZD1480, JAK1_3715, JAK3_7406, MCT1_6447, MCT4_1422, AZD2014, AZD8186, AZD8835, PI3Ka_4409, AZD1208, PLK_6522, RAF_9304, PARP_9495, PARP_0108, PARP_9482, TANK_1366, AZD1332, TTK_3146, SN-38, Pevonedistat, PFI-3, Camptothecin, Staurosporine, Irinotecan, Oxaliplatin, PRIMA-1MET, Niraparib, MK-1775, Dinaciclib, EPZ004777, AZ960, Epirubicin, Cyclophosphamide, Sapitinib, Uprosertib, Alpelisib, Taselisib, EPZ5676, SCH772984, IWP-2, Leflunomide, VE-822, WZ4003, CZC24832, GSK2606414, PFI3, PCI-34051, RVX-208, OTX015, GSK343, ML323, Entospletinib, PRT062607, Ribociclib, Picolinici-acid, AZD5153, CDK9_5576, CDK9_5038, Eg5_9814, ERK_2440, ERK_6604, IRAK4_4710, JAK1_8709, AZD5991, PAK_5339, TAF1_5496, ULK1_4989, VSP34_8731, IGF1R_3801, JAK_8517, Ibrutinib, Zoledronate, Acetalax, Carmustine, Topotecan, Teniposide, Mitoxantrone, Dactinomycin, Fludarabine, Nelarabine, Vincristine, Podophyllotoxin bromide, Dihydrorotenone, Gallibiscoquinazole, Elephantin, Sinularin, Sabutoclax, LY2109761, OF-1, MN-64, KRAS (G12C) Inhibitor-12, BDP-00009066, Buparlisib, Ulixertinib, Venetoclax, ABT737, Afuresertib, AGI-5198, AZD3759, AZD5363, Osimertinib, Cediranib, Ipatasertib, GDC0810, GNE-317, GSK2578215A, I-BRD9, Telomerase Inhibitor IX, MIRA-1, NVP-ADW742, P22077, Savolitinib, UMI-77, WIKI4, WEHI-539, BPD-00008900, BIBR-1532, Pyridostatin, AMG-319, MK-8776, LJI308, AZ6102, GSK591, VE821, and AT13148. In some versions, the methods of predicting the response of a treatment can include predicting the response of a cancer treatment with any one or more of the aforementioned drugs, in any combination

In some versions, the first and/or second genes encompassed by the linear regression predictor models of the invention can comprise any one or more of the following genes, in any combination: A1CF, ABI1, ABL1, ABL2, ACKR3, ACSL3, ACSL6, ACVR1, ACVR2A, AFF1, AFF3, AFF4, AKAP9, AKT1, AKT2, AKT3, ALDH2, ALK, AMER1, ANK1, APC, APOBEC3B, AR, ARAF, ARHGAP26, ARHGAPS, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMPS, BMPR1A, BRAF, BRCA1, BRCA2, BRD3, BRD4, BRIP1, BTG1, BTK, BUB1B, C150RF65, CACNA1D, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, CHCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICER1, DNAJB1, DNM2, DNMT3A, DROSHA, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERCC5, ERG, ESR1, ETNK1, ETV1, ETV4, ETV5, ETV6, EWSR1, EXT1, EXT2, EZH2, EZR, FAM131B, FAM135B, FAM46C, FAM47C, FANCA, FANCC, FANCD2, FANCE, FANCF, FANCG, FAS, FAT1, FAT3, FAT4, FBLN2, FBX011, FBXW7, FCGR2B, FCRL4, FEN1, FES, FEV, FGFR1, FGFR10P, FGFR2, FGFR3, FGFR4, FH, FHIT, FIP1L1, FKBP9, FLCN, FLI1, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA.A, HLF, HMGA1, HMGA2, HMGN2P46, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDHL IDH2, IGF2BP2, IKBKB, IKZF 1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJS, KDMSA, KDMSC, KDM6A, KDR, KDSR, KEAP1, KIAA1549, KIFSB, KIT, KLF4, KLF6, KLK2, KMT2A, KMT2C, KMT2D, KNSTRN, KRAS, KTN1, LARP4B, LASP1, LCK, LCP1, LEF1, LEPROTL1, LIFR, LMNA, LMO1, LMO2, LPP, LRIG3, LRP1B, LSM14A, LYL1, LZTR1, MAF, MAFB, MALAT1, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MKL1, MLF1, MLH1, MLLT1, MLL T10, MLL T11, MLL T3, MLL T6, MN1, MNX1, MPL, MSH2, MSH6, MSI2, MSN, MTCP1, MTOR, MUC1, MUC16, MUC4, MUTYH, MYB, MYC, MYCL, MYCN, MYD88, MYH11, MYH9, MYOSA, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2.1, NONO, NOTCH1, NOTCH2, NPM1, NR4A3, NRAS, NRG1, NSD1, NT5C2, NTHL1, NTRK1, NTRK3, NUMA1, NUP214, NUP98, NUTM1, NUTM2A, NUTM2B, OLIG2, OMD, P2RY8, PABPC1, PAFAH1B2, PALB2, PAX3, PAXS, PAX7, PAX8, PBRM1, PBX1, PCBP1, PCM1, PDCD1LG2, PDE4DIP, PDGFB, PDGFRA, PDGFRB, PERI, PHF6, PHOX2B, PICALM, PIK3CA, PIK3CB, PIK3R1, PIM1, PLAG1, PLCG1, PML, PMS1, PM52, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, SEPT5, SEPT6, SEPT9, SET, SETBP1, SETD1B, SETD2, SF3B1, SFPQ, SFRP4, SGK1, SH2B3, SH3GL1, SIRPA, SIX1, SIX2, SKI, SLC34A2, SLC45A3, SMAD2, SMAD3, SMAD4, SMARCA4, SMARCB 1, SMARCD1, SMARCE1, SMC 1A, SMO, SND1, SOCS1, SOX2, SOX21, SPECC1, SPEN, SPOP, SRC, SRGAP3, SRSF2, SRSF3, SS18, SS18L1, SSX1, SSX2, SSX4, STAG1, STAG2, STAT3, STATSB, STATE, STIL, STK11, STRN, SUFU, SUZ12, SYK, TAF15, TAL1, TAL2, TBL1XR1, TBX3, TCEA1, TCF12, TCF3, TCF7L2, TCL1A, TEC, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRAF7, TRIM24, TRIM27, TRIM33, TRIP11, TRRAP, TSC1, TSC2, TSHR, U2AF1, UBRS, USP44, USP6, USPS, VAV1, VHL, VTI1A, WAS, WIF1, WNK2, WRN, WT1, WWTR1, XPA, XPC, XPO1, YWHAE, ZBTB16, ZCCHC8, ZEB1, ZFHX3, ZMYM3, ZNF331, ZNF384, ZNF429, ZNF479, ZNF521, ZNRF3, and ZRSR2. The gene names provided above are those used in the COSMIC database (cancer.sanger.ac.uk/cosmic) (Tate J G, et al. COSMIC: the catalogue of somatic mutations in cancer. Nucleic Acids Res. 2019;47:D941-D947).

Exemplary predictor intercepts, predictor gene-expression coefficients, and predictor mutation-status coefficient intercepts for the above-mentioned drug treatments and first and second genes are provided in Tables 1A-1I. The columns in Tables 1A-1I provide predictor intercepts, predictor gene-expression coefficients, and predictor mutation-status coefficient for each individual drug, with the name of the relevant drug indicated at the top of the column. Predictor intercepts for each drug are listed in the row entitled “Intercept.” Predictor gene-expression coefficients for the individual genes are provided in the rows labeled with the gene names. Predictor mutation-status coefficients for the individual genes are provided in the rows labeled with the gene names preceded with the prefix “mut_.” In various versions of the invention, the linear regression predictor models of the invention can include as the one or more first genes any one or more of the genes listed in Tables 1A-1I that have a non-zero gene-expression coefficient for a given drug, can include as the one or more second genes any one or more of the genes listed in Tables 1A-1I that have a non-zero mutation-status coefficient for the given drug, can include as the predictor intercept an approximate of the intercept listed in Tables 1A-1I for the given drug, can include as each predictor gene-expression coefficient an approximate of the gene-expression coefficient for one of the one or more first genes listed in Tables 1A-1I for the given drug, and can include as each predictor mutation-status coefficient an approximate of the mutation-status coefficient for one of the one or more second genes listed in Tables 1A-1I for the given drug.

In some versions, the one or more first genes in a given linear regression predictor models for a given drug comprise or consist of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least of 100 of the genes listed in Tables 1A-1I that have a non-zero coefficient for the given drug. In some versions, the one or more first genes in a given linear regression predictor models for a given drug comprise or consist of all the genes listed in Tables 1A-1I that have a non-zero coefficient for the given drug. In some versions, the one or more second genes in a given linear regression predictor models for a given drug comprise of consist of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least of 100 of the genes listed in Tables 1A-1I having a non-zero coefficient for the given drug. In some versions, the one or more second genes in a given linear regression predictor models for a given drug comprise of consist of all the genes listed in Tables 1A-1I having a non-zero coefficient for the given drug.

“Approximate” used with respect to a given value refers to a value within +/−10-fold of the given value, such as +/−9-fold of the given value, +/−8-fold of the given value, +/−7-fold of the given value, +/−6-fold of the given value, +/−5-fold of the given value, +/−4-fold of the given value, +/−3-fold of the given value, +/−2-fold of the given value, +/−1.9-fold of the given value, +/−1.8-fold of the given value, +/−1.7-fold of the given value, +/−1.6-fold of the given value, +/−1.5-fold of the given value, +/−1.4-fold of the given value, +/−1.3-fold of the given value, +/−1.2-fold of the given value, +/−1.1-fold of the given value, +/−1-fold of the given value, +/−0.9-fold of the given value, +/−0.8-fold of the given value, +/−0.7-fold of the given value, +/−0.6-fold of the given value, +/−0.5-fold of the given value, +/−0.4-fold of the given value, +/−0.3-fold of the given value, +/−0.2-fold of the given value, +/−0.1-fold of the given value, +/−0.05-fold of the given value, 0.01-fold of the given value, or 0.001-fold of the given value.

Linear regression predictor models employing the values provided in Tables 1A-1I can be used to predict the response of a cancer patient to any one or more of the drugs listed in Tables 1A-1I in any combination. In some versions, the response of more than one of the drugs listed in Tables 1A-1I is predicted, with a different linear regression predictor model being used for each drug. In some versions, the responses of all of the drugs listed in Tables 1A-1I are predicted, with a different linear regression predictor model being used for each drug. The same gene-expression and mutation status data from a single patient can be used for all the treatment-specific models.

Some aspects of the invention are further directed to administering one or more treatments. The administered treatments are preferably treatments determined to have a favorable treatment response. “Administering” as used herein refers to the application of the treatment to the patient, whether in the form of administering a drug, conducting a surgery, exposing to radiation, etc. The administering preferably ameliorates the disease. “Ameliorates” as used herein refers to the improvement of any aspect of the disease, including any symptom or characteristic thereof, to any degree.

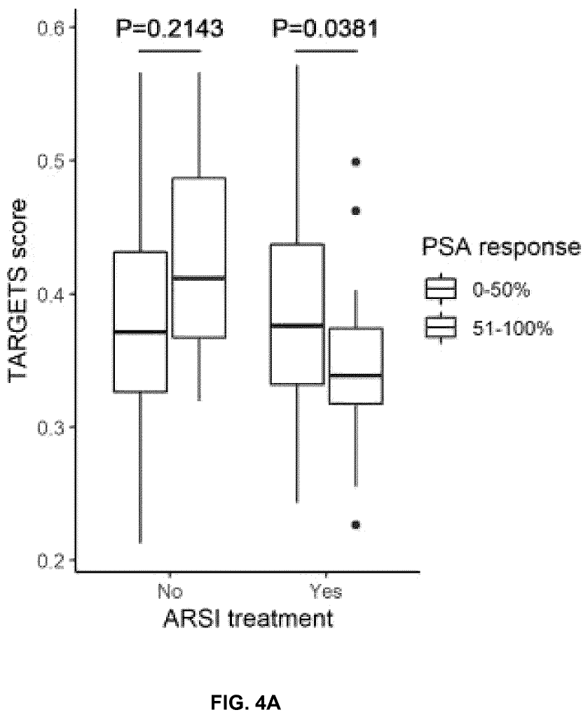

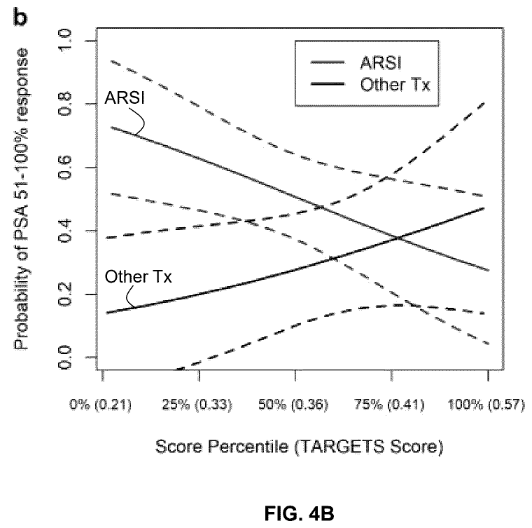

Some embodiments comprise administering the treatment to the patient if the treatment-response score is within a therapeutic range. Some embodiments comprise administering the treatment to the patient if and only if the treatment-response score is within a therapeutic range. The therapeutic range of treatment-response scores for a particular treatment and disease is the range of treatment response scores that indicate at least some ameliorative effect from the treatment. Therapeutic ranges can be determined for a particular treatment and disease by comparing the effect of the particular treatment on the disease with the effects from one or more control interventions and determining the treatment-response scores that provide an enhanced ameliorative effect over the one or more control interventions. The control interventions are understood to include any intervention (or lack thereof) other than the particular treatment, including any one or more treatments other than the particular treatment and/or no treatment at all. An example of such an analysis is shown in FIG. 4B. The particular treatment in this case is treatment with androgen receptor signaling inhibitors (ARSIs), and the disease is metastatic castration-resistant prostate cancer (mCRPC). The analysis includes an interaction plot showing the probability of an ameliorative effect (defined in this particular case as PSA 51-100% response) as a function of the treatment-response scores (defined in this particular case as the TARGETS scores) in the patients treated with an ARSI vs. other treatments. This type of plot can be generated for any treatment-response score for any treatment and disease. In order to transform the treatment-response scores into predicted response rates (or any other clinical endpoint such as progression, death, etc.) the difference between the line representing the treatment-response scores of the treatment and the line representing the treatment response scores of the control intervention(s) can be taken. The magnitude of this difference can represent the predicted benefit in terms of response rate (or any other clinical endpoint). For example, for the ARSI response score shown in FIG. 4B, the curves cross around the 75th percentile. Thus, at a score below this, a patient would be predicted to more likely respond to an ARSI than to some other treatment (including no treatment). At a score around the 65^{th} percentile, the degree of benefit increases to where an ARSI is predicted to have approximately >10% benefit in terms of response rate compared to an alternative treatment. The exact threshold for determining whether or not to give a particular treatment in any particular case may incorporate the information from these predictions (i.e. response rate with vs. without the particular treatment), as well as other variables such as side effects, cost, patient preferences, in order to make a shared decision between a physician or a patient if a particular treatment is the best option for a patient.

In some versions of the invention, the treatment may be administered to a patient if the treatment-response score indicates a response rate equal to or greater than a threshold response rate, wherein the threshold response rate indicates a probability of having an ameliorative effect over a control intervention, in the manner described above. In various versions of the invention, the threshold response rate can be greater than 0, greater than 0.01, greater than 0.05, greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.9.5, or 1.

For administration of drugs, such as the drugs listed above and in Tables 1A-1I, the drugs can be administered in therapeutically effective amounts either alone or as part of a pharmaceutical composition. “Drug” as used herein refers to a substance that has a biological effect on a cell, tissue, or organism. Examples of drugs include small molecule compounds, biologics, and other substances. “Therapeutically effective amount” refers to an amount that ameliorates the disease. The pharmaceutical compositions can comprise at least one drug and one or more pharmaceutically acceptable carriers, diluents, or excipients. The routes of administration include, but are not limited to, oral administration, intravitreal administration, rectal administration, parenteral, intravenous administration, intraperitoneal administration, intramuscular administration, inhalation, transmucosal administration, pulmonary administration, intestinal administration, subcutaneous administration, intramedullary administration, intrathecal administration, direct intraventricular, intranasal administration, topical administration, or ophthalmic administration.

In certain embodiments, the drugs are administered locally, while in other embodiments the drugs are administered systemically. Local administration includes, but is not limited to, injection into an organ, optionally in a depot or sustained release formulation. Systemic administration includes, but is not limited to, oral administration, or intravenous administration. In other embodiments, the drugs are administered in a targeted drug delivery system, such as, by way of example only, in a liposome coated with organ-specific antibody. The liposome is targeted to and taken up selectively by the organ. In other embodiments, the drugs are administered in the form of a rapid release formulation, while in other embodiments, the drugs are administered in the form of an extended release formulation. In other embodiments, the drugs are administered in the form of an intermediate release formulation.

The therapeutically effective amount will vary depending on, among others, the disease indicated, the severity of the disease, the age and relative health of the subject, the potency of the drug administered, the route of administration, and the treatment desired.

The pharmaceutical compositions of the present invention can be in unit dosage form. Exemplary unit dosages include about 1-1000 mg of drug(s) for a subject of about 50-70 kg, or about 1-500 mg or about 1-250 mg or about 1-150 mg or about 0.5-100 mg, or about 1-50 mg of active ingredients. The therapeutically effective dosage of a drug is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The drugs can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration. Exemplary therapeutically effective amounts include between about 0.1-500 mg/kg, or between about 1-100 mg/kg.

Processes for the preparation of pharmaceutical compositions can include admixing a drug with one or more pharmaceutically acceptable carriers, diluents or excipients. In certain embodiments, the pharmaceutical compositions comprise a drug in free form or in a pharmaceutically acceptable salt or solvate form. In certain embodiments, the pharmaceutical compositions are manufactured by mixing, dissolving, granulating, levigating, emulsifying, encapsulating, entrapping or compression processes, and/or coating methods. In other embodiments, such compositions optionally contain excipients, such as preserving, stabilizing, wetting or emulsifying agents, solution promoters, salts for regulating the osmotic pressure and/or buffers. In other embodiments, pharmaceutical compositions are sterilized.

In certain embodiments, the drugs are administered orally as discrete dosage forms, wherein such dosage forms include, but are not limited to, capsules, gelatin capsules, caplets, tablets, chewable tablets, powders, pills, granules, liquids, gels, syrups, flavored syrups, elixirs, slurries, solutions or suspensions in aqueous or non-aqueous liquids, edible foams or whips, and oil-in-water liquid emulsions or water-in-oil liquid emulsions. The capsules, gelatin capsules, caplets, tablets, lozenges, chewable tablets, powders or granules are prepared by admixing at least drug together with at least one excipient using conventional pharmaceutical compounding techniques. Non-limiting examples of excipients used in oral dosage forms described herein include, but are not limited to, binders, fillers, disintegrants, lubricants, absorbents, colorants, flavors, preservatives and sweeteners.

Non-limiting examples of such binders include, but are not limited to, corn starch, potato starch, starch paste, pre-gelatinized starch, or other starches, sugars, gelatin, natural and synthetic gums such as acacia, sodium alginate, alginic acid, other alginates, tragacanth, guar gum, cellulose and its derivatives (by way of example only, ethyl cellulose, cellulose acetate, carboxymethyl cellulose calcium, sodium carboxymethylcellulose, methyl cellulose, hydroxypropyl methylcellulose and microcrystalline cellulose), magnesium aluminum silicate, polyvinyl pyrrolidone and combinations thereof.

Non-limiting examples of such fillers include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein are present in from about 50 to about 99 weight percent of the pharmaceutical composition or dosage form.

Non-limiting examples of such disintegrants include, but are not limited to, agar-agar, alginic acid, sodium alginate, calcium carbonate, sodium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and combinations thereof. In certain embodiments, the amount of disintegrant used in the pharmaceutical compositions provided herein is from about 0.5 to about 15 weight percent of disintegrant, while in other embodiments the amount is from about 1 to about 5 weight percent of disintegrant.

Non-limiting examples of such lubricants include, but are not limited to, sodium stearate, calcium stearate, magnesium stearate, stearic acid, mineral oil, light mineral oil, glycerin, sorbitol, mannitol, polyethylene glycol, other glycols, sodium lauryl sulfate, talc, hydrogenated vegetable oil (by way of example only, peanut oil, cottonseed oil, sunflower oil, sesame oil, olive oil, corn oil, and soybean oil), zinc stearate, sodium oleate, ethyl oleate, ethyl laureate, agar, silica, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and combinations thereof. In certain embodiments, the amount of lubricants used in the pharmaceutical compositions provided herein is in an amount of less than about 1 weight percent of the pharmaceutical compositions or dosage forms.

Non-limiting examples of such diluents include, but are not limited to, lactose, dextrose, sucrose, mannitol, sorbitol, cellulose, glycine or combinations thereof.

In certain embodiments, tablets and capsules are prepared by uniformly admixing a drug with a liquid carrier, finely divided solid carriers, or both, and then shaping the product into the desired presentation if necessary. In certain embodiments, tablets are prepared by compression. In other embodiments, tablets are prepared by molding.

In certain embodiments, a drug is orally administered as a controlled release dosage form. Such oral dosage forms may be either film coated or enteric coated according to methods known in the art. Such dosage forms are used to provide slow or controlled-release drug forms. Controlled release is obtained using, for example, hydroxypropylmethyl cellulose, other polymer matrices, gels, permeable membranes, osmotic systems, multilayer coatings, microparticles, liposomes, microspheres, or a combination thereof. In certain embodiments, controlled-release dosage forms are used to extend activity of the drug, reduce dosage frequency, and increase patient compliance.

Administration of drugs as oral fluids such as solution, syrups and elixirs are prepared in unit dosage forms such that a given quantity of solution, syrup, or elixir contains a predetermined amount of drug. Syrups are prepared by dissolving the drug in a suitably flavored aqueous solution, while elixirs are prepared through the use of a non-toxic alcoholic vehicle. Suspensions are formulated by dispersing the drug in a non-toxic vehicle. Non-limiting examples of excipients used in as oral fluids for oral administration include, but are not limited to, solubilizers, emulsifiers, flavoring agents, preservatives, and coloring agents. Non-limiting examples of solubilizers and emulsifiers include, but are not limited to, water, glycols, oils, alcohols, ethoxylated isostearyl alcohols and polyoxy ethylene sorbitol ethers. Non-limiting examples of preservatives include, but are not limited to, sodium benzoate. Non-limiting examples of flavoring agents include, but are not limited to, peppermint oil or natural sweeteners or saccharin or other artificial sweeteners.

In certain embodiments, the drugs administered parenterally by various routes including, but not limited to, subcutaneous, intravenous (including bolus injection), intramuscular, and intraarterial.

Such parenteral dosage forms are administered in the form of sterile or sterilizable injectable solutions, suspensions, dry and/or lyophilized products ready to be dissolved or suspended in a pharmaceutically acceptable vehicle for injection (reconstitutable powders) and emulsions. Vehicles used in such dosage forms include, but are not limited to, Water for Injection USP; aqueous vehicles such as, but not limited to, Sodium Chloride Injection, physiological saline buffer, Ringers Injection solution, Dextrose Injection, Dextrose and Sodium Chloride Injection, and Lactated Ringer's Injection solution; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In certain embodiments, the drugs are parenterally administered by bolus injection. In other embodiments, are parenterally administered by continuous infusion. Formulations for injection are presented in unit dosage form, by way of example only, in ampoules or formulations for injection are presented in multi-dose containers, with an added preservative. The compositions may take such forms as suspensions, solutions or emulsions in oily or aqueous vehicles, and may contain formulatory agents such as suspending, stabilizing and/or dispersing agents.

In certain embodiments the drugs are administered transdermally. Such transdermal dosage forms include “reservoir type” or “matrix type” patches, which are applied to the skin and worn for a specific period of time to permit the penetration of a desired amount of a drug. By way of example only, such transdermal devices are in the form of a bandage comprising a backing member, a reservoir containing the drug optionally with carriers, optionally a rate controlling barrier to deliver the drug to the skin of the host at a controlled and predetermined rate over a prolonged period of time, and means to secure the device to the skin. In other embodiments, matrix transdermal formulations are used. In certain embodiments transdermal administration is used to provide continuous, while in other embodiments transdermal administration is used to provide discontinuous infusion of a drug in controlled amounts.

In certain embodiments, the rate of absorption is slowed by using rate-controlling membranes or by trapping the drug within a polymer matrix or gel. In certain embodiments, transdermal delivery is via a transdermal patch.

Formulations for transdermal delivery of drugs include a drug, a carrier, and an optional diluent. A carrier includes, but is not limited to, absorbable pharmacologically acceptable solvents to assist passage through the skin of the host, such as water, acetone, ethanol, ethylene glycol, propylene glycol, butane-1,3-diol, isopropyl myristate, isopropyl palmitate, mineral oil, and combinations thereof.

In certain embodiments, such transdermal delivery systems include penetration enhancers to assist in delivering the drugs to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

In some embodiments, transdermal delivery of drugs is accomplished by means of iontophoretic patches and the like.

In certain embodiments, drugs are administered by topical application of pharmaceutical composition containing drugs in the form of lotions, gels, ointments solutions, emulsions, suspensions or creams. Suitable formulations for topical application to the skin are aqueous solutions, ointments, creams or gels, while formulations for ophthalmic administration are aqueous solutions. Such formulations optionally contain solubilizers, stabilizers, tonicity enhancing agents, buffers and preservatives.

Such topical formulations include at least one carrier, and optionally at least one diluent. Such carriers and diluents include, but are not limited to, water, acetone, ethanol, ethylene glycol, propylene glycol, butane-1,3-diol, isopropyl myristate, isopropyl palmitate, mineral oil, and combinations thereof.

In certain embodiments, such topical formulations include penetration enhancers to assist in delivering drugs to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

In certain embodiments, drugs are administered by inhalation. Dosage forms for inhaled administration are formulated as aerosols or dry powders. Aerosol formulations for inhalation administration comprise a solution or fine suspension of at least one drug in a pharmaceutically acceptable aqueous or non-aqueous solvent. In addition, such pharmaceutical compositions optionally comprise a powder base such as lactose, glucose, trehalose, mannitol or starch, and optionally a performance modifier such as L-leucine or another amino acid, and/or metals salts of stearic acid such as magnesium or calcium stearate.

In certain embodiments, drugs administered directly to the lung by inhalation using a Metered Dose Inhaler (“MDI”), which utilizes canisters that contain a suitable low boiling propellant, e.g., dichlorodifluoromethane, trichlorofluoromethane, di chlorotetrafluoroethane, carbon dioxide or other suitable gas, or a Dry Powder Inhaler (DPI) device which uses a burst of gas to create a cloud of dry powder inside a container, which is then be inhaled by the patient. In certain embodiments, capsules and cartridges of gelatin for use in an inhaler or insufflator are formulated containing a powder mixture of a drug and a powder base such as lactose or starch. In certain embodiments, drugs are delivered to the lung using a liquid spray device, wherein such devices use extremely small nozzle holes to aerosolize liquid drug formulations that can then be directly inhaled into the lung. In other embodiments, drugs are delivered to the lung using a nebulizer device, wherein a nebulizers creates an aerosols of liquid drug formulations by using ultrasonic energy to form fine particles that can be readily inhaled. In other embodiments, drugs are delivered to the lung using an electrohydrodynamic (“EHD”) aerosol device wherein such EHD aerosol devices use electrical energy to aerosolize liquid drug solutions or suspensions.

In certain embodiments drugs are administered nasally. The dosage forms for nasal administration are formulated as aerosols, solutions, drops, gels or dry powders.

In certain embodiments, drugs are administered rectally in the form of suppositories, enemas, retention enemas ointment, creams rectal foams or rectal gels. In certain embodiments such suppositories are prepared from fatty emulsions or suspensions, cocoa butter or other glycerides.

In certain embodiments, drugs are formulated as a depot preparation. Such long acting formulations are administered by implantation (for example subcutaneously or intramuscularly) or by intramuscular injection. In certain embodiments, such formulations include polymeric or hydrophobic materials (for example, as an emulsion in an acceptable oil) or ion exchange resins, or as sparingly soluble derivatives, for example, as a sparingly soluble salt.

In certain embodiments injectable depot forms are made by forming microencapsulated matrices of the drugs in biodegradable polymers. The rate of drug release is controlled by varying the ratio of drug to polymer and the nature of the particular polymer employed. In other embodiments, depot injectable formulations are prepared by entrapping the drugs in liposomes or microemulsions.

In certain embodiments, drugs are ophthalmically administered to the eye. Administration to the eye generally results in direct contact of the agents with the cornea, through which at least a portion of the administered agents pass.

Ophthalmic administration, as used herein, includes, but is not limited to, topical administration, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the formix). In certain embodiments, the drugs are formulated as an ophthalmic composition and are administered topically to the eye. Such topically administered ophthalmic compositions include, but are not limited to, solutions, suspensions, gels or ointments.

In certain embodiments, the pharmaceutical compositions used for ophthalmic administration take the form of a liquid where the drugs are present in solution, in suspension or both. In some embodiments, a liquid composition includes a gel formulation. In other embodiments, the liquid composition is aqueous. In other embodiments, such liquid compositions take the form of an ointment. In certain embodiments pharmaceutical compositions containing at least one drug are administered ophthalmically as eye drops formulated as aqueous solutions that optionally contain solubilizers, stabilizers, tonicity enhancing agents, buffers and preservatives. A desired dosage is administered via a known number of drops into the eye.

In certain embodiments the aqueous compositions have an ophthalmically acceptable pH and osmolality. In certain embodiments the aqueous compositions include one or more ophthalmically acceptable pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### SUMMARY

We are now in an era of molecular medicine, where specific DNA alterations can be used to identify patients who will respond to specific drugs. However, there are only a handful of clinically used predictive biomarkers in oncology. Herein, we describe an approach utilizing in vitro DNA and RNA sequencing and drug response data to create TreAtment Response Generalized Elastic-neT Signatures (TARGETS). We trained TARGETS drug response models using Elastic-Net regression in the publicly available Genomics of Drug Sensitivity in Cancer (GDSC) database. Models were then validated on additional in-vitro data from the Cancer Cell Line Encyclopedia (CCLE), and on clinical samples from The Cancer Genome Atlas (TCGA) and Stand Up to Cancer/Prostate Cancer Foundation West Coast Prostate Cancer Dream Team (WCDT). First, we demonstrated that all TARGETS models successfully predicted treatment response in the separate in-vitro CCLE treatment response dataset. Next, we evaluated all FDA-approved biomarker-based cancer drug indications in TCGA and demonstrated that TARGETS predictions were concordant with established clinical indications. Finally, we performed independent clinical validation in the WCDT and found that the TARGETS AR signaling inhibitors (ARSI) signature successfully predicted clinical treatment response in metastatic castration-resistant prostate cancer with a statistically significant interaction between the TARGETS score and PSA response (p=0.0252). TARGETS represents a pan-cancer, platform-independent approach to predict response to oncologic therapies and could be used as a tool to better select patients for existing; therapies as well as identify new indications for testing in prospective clinical trials.

### Introduction

Treatment decisions for cancer patients have historically depended on the tumor location and histologic appearance. However, response is often heterogeneous within the same tumor type′. Molecular diversity is fundamental to a cancer's ability to evade endogenous and exogenous tumor control strategies, and there is a great need to incorporate an understanding of this diversity into the management of all cancer patients. Advances in next-generation sequencing have ushered in a personalized treatment approach that can improve tumor control and decrease side effects compared to the traditional one-size-fits-all approach.

Multiple anti-neoplastic therapies have now been paired with predictive biomarkers for making treatment decisions. This approach has been particularly successful with targeted drug therapies. The first successful examples include Imatinib for chronic myelogenous leukemia patients with the BCR-ABL fusion 2 and Trastuzumab for HER2-positive breast cancer patients′. Since the approval of these agents 20 years ago, the FDA has approved dozens of different targeted therapies, with the number increasing rapidly every year. However, even among these targeted therapies and among patients who have a mutation known to confer increased sensitivity to the therapy, treatment outcomes can still be heterogeneous. For example, even among non-small cell lung cancer (NSCLC) patients with classic EGFR mutations, where exon 19 deletions and L858R exon 21 point mutations account for 90% of EGFR mutations, response rates have ranged from 58 to 85% in phase Iib/III clinical trials evaluating anti-EGFR tyrosine kinase inhibitors (e.g., Erlotinib, Gefitinib, Afatinib, Osimertinib)⁴⁻¹¹.

A contributing factor to variability in treatment response is the complex and often compound nature of cancer gene alterations. Multiple mutations and gene expression differences likely modulate response, but many of the relevant changes are challenging to identify. We hypothesized that next-generation DNA and RNA sequencing techniques paired with modern computational modeling could identify gene signatures that would better capture this heterogeneity. Rather than relying on the presence or absence of a single genetic variant, we instead model treatment predictions based on a broad spectrum of genomic variant and expression data. To do this, we have leveraged an existing large-scale in-vitro database to train TreAtment Response Generalized Elastic-neT Signatures (TARGETS). We then validated these results on three independent cohorts. First, we showed concordant drug-response predictions in an external in-vitro database. Second, we demonstrated that our predictions were concordant with known FDA biomarkers-drug indications in a large cohort of sequenced tumors. Third, we validated TARGETS as a predictive biomarker of androgen receptor signaling inhibitor (ARSI) response in a unique dataset of metastatic prostate cancer patients. Finally, we evaluated the utility of TARGETS as a tool for targeted hypothesis generation in identifying new drug indications. This pan-cancer, platform-independent approach can be used to better identify responders vs. non-responders and could potentially identify new patient populations which would benefit from specific treatments.

### Methods

### Literature Review of FDA Approved Somatic Biomarker Indications in Cancer

To establish a comprehensive list of all clinically approved biomarker-drug combinations to analyze in this study, we obtained a list of United States Food and Drug Administration (FDA) pharmacogenomic indications (World Wide Web at fda.gov/drugs/science-and-research-drug s/table-pharmacogenomic-biomarkers-drug-labeling, version dated 5 Feb. 2020; Table 2). In addition to the biomarker-drug combinations in the FDA list, we also examined clinically utilized MGMT promoter methylation with Temozolomide in glioblastoma²⁰⁻²³ and homologous recombination deficiency with Olaparib in prostate cancer²⁵. While PARP inhibitors such as Olaparib are indicated for both HRD and non-HRD ovarian cancers' and are also indicated for germline BRCA1/2 mutant breast cancer, germline variants are restricted data in the TCGA, and our focus was on somatic variants, so these germline indications were not assessed. EML4-ALK and ROS1 fusions were called using the Jackson Laboratory Tumor Fusion Gene Data Portal (World Wide Web at tumorfusions.org)⁶¹. As fusion partners for ROS1 are less well defined, only ROS1 fusions confirmed by WGS were included. ER, PR, HER2 positivity, MGMT promoter methylation, and FLT3 mutation were defined by the TCGA phenotypic data. All other mutations were defined by the sequencing data. EGFR staining was not available, and so EGFR positivity was defined as greater than median EGFR expression, based on literature supporting a range of EGFR positivity of 25-82% in colorectal cancer⁶².

TABLE 2
FDA pharmacogenomic biomarkers.

| | | | Cancer | Drug | |
|---|---|---|---|---|---|
| | | | Site in | Included in | |
| Drug | Target | Cancer Site | TCGA | GDSC | Biomarker |
| Abemaciclib | ESR (Hormone Receptor), | BRCA | TRUE | FALSE | |
| | ERBB2 (HER2) | | | | |
| Ado- | ERBB2 (HER2) | BRCA | TRUE | FALSE | |
| Trastuzumab | | | | | |
| Emtansine | | | | | |
| Afatinib | EGFR | NSCLC | TRUE | TRUE | EGFR |
| | | | | | mutant |
| Alectinib | ALK | NSCLC | TRUE | TRUE | EML4-ALK |
| | | | | | fusion |
| Alpelisib | ERBB2 (HER2), ESR | BRCA | TRUE | TRUE | PIK3CA |
| | (Hormone Receptor), | | | | mutant |
| | PIK3CA | | | | |
| Anastrozole | ESR, PGR (Hormone | BRCA | TRUE | FALSE | |
| | Receptor) | | | | |
| Arsenic Trioxide | PML-RARA | APL | FALSE | FALSE | |
| Atezolizumab | CD274 (PD-L1), Gene | Urothelial carcinoma, | TRUE | FALSE | |
| | Signature (T-effector), | NSCLC, ES-SCLC, | | | |
| | EGFR, ALK | BRCA | | | |
| Avelumab | CD274 (PD-L1) | Merkel cell carcinoma, | TRUE | FALSE | |
| | | urothelial carcinoma, | | | |
| | | RCC | | | |
| Belinostat | UGT1A1 | Peripheral T-cell | FALSE | TRUE | |
| | | lymphoma | | | |
| Binimetinib | BRAF, UGT1A1 | Melanoma | TRUE | FALSE | |
| Blinatumomab | BCR-ABL1 (Philadelphia | ALL | FALSE | FALSE | |
| | chromosome) | | | | |
| Bosutinib | BCR-ABL1 (Philadelphia | CML | FALSE | TRUE | |
| | chromosome) | | | | |
| Brentuximab | ALK, TNFRSF8 (CD30) | Various Lymphomas | FALSE | FALSE | |
| Vedotin | | | | | |
| Brigatinib | ALK | NSCLC | TRUE | FALSE | |
| Busulfan | BCR-ABL1 (Philadelphia | CML | FALSE | FALSE | |
| | chromosome) | | | | |
| Cabozantinib | RET | RCC, HCC | TRUE | TRUE | |
| Capecitabine | DPYD | CRC, BRCA | TRUE | FALSE | |
| Ceritinib | ALK | NSCLC | TRUE | FALSE | |
| Cetuximab | EGFR, RAS | HNSCC, CRC | TRUE | TRUE | EGFR |
| | | | | | positive & |
| | | | | | KRAS wt |
| | | | | | CRC |
| Cisplatin | TPMT | Testicular, ovarian and | TRUE | TRUE | |
| | | transitional cell bladder | | | |
| | | cancer | | | |
| Cobimetinib | BRAF | Melanoma | TRUE | FALSE | |
| Crizotinib | ALK, ROS1 | NSCLC | TRUE | TRUE | EML4- |
| | | | | | ALK, ROS1 |
| | | | | | fusion |
| Dabrafenib | BRAF, G6PD, RAS | Melanoma, NSCLC, | TRUE | TRUE | BRAF |
| | | anaplastic thyroid cancer | | | V600E |
| | | | | | mutant |
| Dacomitinib | EGFR | NSCLC | TRUE | FALSE | |
| Dasatinib | BCR-ABL1 (Philadelphia | CML and ALL | FALSE | TRUE | |
| | chromosome) | | | | |
| Denileukin | IL2RA (CD25 antigen) | Cutaneous T Cell | FALSE | FALSE | |
| Diftitox | | Lymphoma | | | |
| Dinutuximab | MYCN | Neuroblastoma | FALSE | FALSE | |
| Docetaxel | ESR, PGR (Hormone | Breast, gastric/GE | TRUE | TRUE | |
| | Receptor) | junction Cancer, | | | |
| | | NSCLC, metastatic | | | |
| | | castration resistant | | | |
| | | prostate cancer, HNSCC | | | |
| Durvalumab | CD274 (PD-L1) | urothelial carcinoma, | TRUE | FALSE | |
| | | NSCLC, ES-SCLC | | | |
| Duvelisib | Chromosome 17p | CLL, SLL, Follicular | FALSE | FALSE | |
| | | Lymphoma | | | |
| Enasidenib | IDH2 | AML | TRUE | FALSE | |
| Encorafenib | BRAF | Melanoma, CRC | TRUE | FALSE | |
| Enfortumab | NECTIN4 | Urothelial cancer | TRUE | FALSE | |
| Vedotin-ejfv | | | | | |
| Entrectinib | ROS1, NTRK | NSCLC, solid tumors | TRUE | FALSE | |
| | | with a NTRK gene | | | |
| | | fusion | | | |
| Erdafitinib | FGFR, CYP2C9 | Urothelial carcinoma | TRUE | FALSE | |
| Eribulin | ERBB2 (HER2), ESR, | BRCA | TRUE | FALSE | |
| | PGR (Hormone Receptor) | | | | |
| Erlotinib | EGFR | NSCLC and pancreatic | TRUE | TRUE | EGFR |
| | | cancer | | | mutant |
| | | | | | NSCLC |
| Everolimus | ERBB2 (HER2), ESR | Pancreatic | TRUE | FALSE | |
| | (Hormone Receptor) | neuroendocrine tumors, | | | |
| | | RCC, subependymal | | | |
| | | giant cell astrocytoma, | | | |
| | | renal angiomyolipoma | | | |
| Exemestane | ESR, PGR (Hormone | BRCA | TRUE | FALSE | |
| | Receptor) | | | | |
| Fam- | ERBB2 (HER2) | BRCA | TRUE | FALSE | |
| Trastuzumab | | | | | |
| Deruxtecan-nxki | | | | | |
| Fluorouracil | DPYD | CRC, BRCA, gastric | TRUE | FALSE | |
| | | and pancreas cancer | | | |
| Flutamide | G6PD | PRAD | TRUE | FALSE | |
| Fulvestrant | ERBB2 (HER2), ESR, | BRCA | TRUE | TRUE | ER/PR+ |
| | PGR (Hormone Receptor) | | | | |
| Gefitinib | EGFR, CYP2D6 | NSCLC | TRUE | TRUE | EGFR |
| | | | | | mutant |
| Gilteritinib | FLT3 | AML | TRUE | FALSE | |
| Goserelin | ESR, PGR (Hormone | PRAD | TRUE | FALSE | |
| | Receptor) | | | | |
| Ibrutinib | Chromosome 17p, | Mantle cell and marginal | FALSE | TRUE | |
| | Chromosome 11q | zone lymphoma, CLL, | | | |
| | | SLL, Waldenstroms | | | |
| | | macroglobulinemia | | | |
| Imatinib | KIT, BCR-ABL1 | CML, ALL, | FALSE | TRUE | |
| | (Philadelphia | myelodysplastic/myelo- | | | |
| | chromosome), PDGFRB, | proliferative diseases, | | | |
| | FIP1L1-PDGFRA | systemic mastocytosis, | | | |
| | | hypereosinophilic | | | |
| | | syndrome and/or chronic | | | |
| | | eosinophilic leukemia, | | | |
| | | dermatofibrosarcoma | | | |
| | | protuberans, GIST | | | |
| Inotuzumab | BCR-ABL1 (Philadelphia | ALL | FALSE | FALSE | |
| Ozogamicin | chromosome) | | | | |
| Ipilimumab | HLA-A, Microsatellite | Melanoma, RCC, CRC, | TRUE | FALSE | |
| | Instability, Mismatch | HCC | | | |
| | Repair | | | | |
| Irinotecan | UGT1A1 | CRC | TRUE | TRUE | |
| Ivosidenib | IDH1 | AML | TRUE | FALSE | |
| Ixabepilone | ERBB2 (HER2), ESR, | BRCA | TRUE | FALSE | |
| | PGR (Hormone Receptor) | | | | |
| Lapatinib | ERBB2 (HER2), ESR, | BRCA | TRUE | TRUE | HER2+ |
| | PGR (Hormone Receptor), | | | | |
| | HLA-DQA1, HLA- | | | | |
| | DRB1 | | | | |
| Larotrectinib | NTRK | Solid tumors with an | TRUE | FALSE | |
| | | NTRK gene fusion | | | |
| Lenvatinib | Microsatellite Instability, | Differentiated thyroid | TRUE | FALSE | |
| | Mismatch Repair | cancer, RCC, HCC, | | | |
| | | endometrial carcinoma | | | |
| Letrozole | ESR, PGR (Hormone | BRCA | TRUE | FALSE | |
| | Receptor) | | | | |
| Lorlatinib | ALK, ROS1 | NSCLC | TRUE | FALSE | |
| Mercaptopurine | TPMT, NUDT15 | ALL | FALSE | FALSE | |
| Midostaurin | FLT3, NPM1, KIT | AML, systemic | TRUE | TRUE | FLT3+ |
| | | mastocytosis | | | |
| Neratinib | ERBB2 (HER2), ESR, | BRCA | TRUE | FALSE | |
| | PGR (Hormone Receptor) | | | | |
| Nilotinib | BCR-ABL1 (Philadelphia | CML | FALSE | TRUE | |
| | chromosome), UGT1A1 | | | | |
| Niraparib | BRCA | Epithelial ovarian, | TRUE | TRUE | |
| | | fallopian tube, or | | | |
| | | primary peritoneal | | | |
| | | cancer | | | |
| Nivolumab | BRAF, CD274 (PD-L1), | Melanoma, NSCLC, | TRUE | FALSE | |
| | Microsatellite Instability, | SCLC, RCC, Classical | | | |
| | Mismatch Repair, EGFR, | Hodkin's Lymphoma, | | | |
| | ALK | HNSCC, CRC, | | | |
| | | urothelial carcinoma, | | | |
| | | HCC | | | |
| Obinutuzumab | MS4A1 (CD20 antigen) | CLL, follicular | FALSE | FALSE | |
| | | lymphoma | | | |
| Olaparib | BRCA, ERBB2 (HER2), | Pancreatic | TRUE | TRUE | germline |
| | ESR, PGR (Hormone | adenocarcinoma, | | | BRCA |
| | Receptor) | BRCA, epithelial | | | mutant |
| | | ovarian, fallopian tube or | | | BRCA or |
| | | primary peritoneal | | | pancreatic |
| | | cancer | | | cancer |
| Olaratumab | PDGFRA | STS | TRUE | FALSE | |
| Omacetaxine | BCR-ABL1 (Philadelphia | CML | FALSE | FALSE | |
| | chromosome) | | | | |
| Osimertinib | EGFR | NSCLC | TRUE | TRUE | EGFR |
| | | | | | mutant |
| Palbociclib | ESR (Hormone Receptor), | BRCA | TRUE | TRUE | ER/PR+ & |
| | ERBB2 (HER2) | | | | HER2− |
| Panitumumab | EGFR, RAS | CRC | TRUE | FALSE | |
| Pazopanib | UGT1A1, HLA-B | RCC, STS | TRUE | TRUE | |
| Pembrolizumab | BRAF, CD274 (PD-L1), | Melanoma, NSCLC, | TRUE | FALSE | |
| | Microsatellite Instability, | HNSCC, classical | | | |
| | Mismatch Repair, EGFR, | Hodgkin lymphoma, | | | |
| | ALK | mediastinal large B cell | | | |
| | | lymphoma, urothelial | | | |
| | | carcinoma, bladder | | | |
| | | cancer, MSI high or | | | |
| | | MMR deficient solid | | | |
| | | tumor, gastric/GE | | | |
| | | junction cancer, cervical | | | |
| | | cancer, HCC, Merkel | | | |
| | | Cell Carcinoma, RCC, | | | |
| | | esophageal SCC, | | | |
| | | endometrial carcinoma | | | |
| Pertuzumab | ERBB2 (HER2), ESR, | BRCA | TRUE | FALSE | |
| | PGR (Hormone Receptor) | | | | |
| Ponatinib | BCR-ABL1 (Philadelphia | CML, ALL | FALSE | TRUE | |
| | chromosome) | | | | |
| Ramucirumab | EGFR, RAS | Gastric/GE junction | TRUE | FALSE | |
| | | adenocarcinoma, | | | |
| | | NSCLC, CRC | | | |
| Rasburicase | G6PD, CYB5R | Tumor lysis treatment | FALSE | FALSE | |
| Regorafenib | RAS | CRC, GIST | TRUE | FALSE | |
| Ribociclib | ESR, PGR (Hormone | BRCA | TRUE | TRUE | ER/PR+ & |
| | Receptor), ERBB2 | | | | HER2− |
| | (HER2) | | | | |
| Rituximab | MS4A1 (CD20 antigen) | Low grade or follicular | FALSE | FALSE | |
| | | NHL, CLL | | | |
| Rucaparib | BRCA, CYP2D6, | BRCA | TRUE | TRUE | germline |
| | CYP1A2, Homologous | | | | BRCA |
| | Recombination Deficiency | | | | mutant |
| Talazoparib | BRCA, ERBB2 (HER2) | BRCA | TRUE | TRUE | germline |
| | | | | | BRCA |
| | | | | | mutant |
| Tamoxifen | ESR, PGR (Hormone | BRCA | TRUE | TRUE | ER/PR+ |
| | Receptor), F5 (Factor V | | | | |
| | Leiden), F2 (Prothrombin), | | | | |
| | CYP2D6 | | | | |
| Thioguanine | TPMT, NUDT15 | Acute nonlymphocytic | TRUE | FALSE | |
| | | leukemias | | | |
| Tipiracil and | ERBB2 (HER2), RAS | CRC and gastric/GE | TRUE | FALSE | |
| Trifluridine | | junction | | | |
| | | adenocarcinoma | | | |
| Toremifene | ESR (Hormone Receptor) | BRCA | TRUE | FALSE | |
| Trametinib | BRAF, G6PD, RAS | Melanoma, NSCLC | TRUE | TRUE | BRAF |
| | | | | | V600E/K |
| | | | | | mutant |
| Trastuzumab | ERBB2 (HER2), ESR, | BRCA and Gastric/GE | TRUE | FALSE | |
| | PGR (Hormone Receptor) | junction | | | |
| | | adenocarcinoma | | | |
| Tretinoin | PML-RARA | APML | FALSE | TRUE | |
| Vemurafenib | BRAF, RAS | Melanoma | TRUE | FALSE | |
| Venetoclax | Chromosome 17p, | CLL, SLL, AML | TRUE | TRUE | |
| | Chromosome 11q, TP53, | | | | |
| | IDH1/2, IGH, NPM1, | | | | |
| | FLT3 | | | | |
| Vincristine | BCR-ABL1 (Philadelphia | Acute leukemia, | FALSE | TRUE | |
| | chromosome) | Hodgkin and NH | | | |
| | | Lymphoma, | | | |
| | | rhabdomyosarcoma, | | | |
| | | neuroblastoma, Wilm's | | | |
| | | tumor | | | |

### Training in GDSC

Processed mutation calls and RNA-seq FPKM gene expression data on cancer cell lines publicly available through the GDSC were downloaded from the GDSC website (World Wide Web at cancerrxgene.org)¹². Mutations were coded as “present” only if they affected the protein-coding region of a gene (i.e., excluding silent, intronic, and inter-genic mutations), otherwise, they were coded as “not present”. Gene expression was Log 2 transformed, scaled to the median of the cohort, and treated as a continuous variable. We filtered variant and expression data to focus on the 702 COSMIC cancer genes present on all platforms in the training and validation cohorts¹⁵. The GDSC database contains IC₅₀ information for 449 drugs across 982 cell lines and DNA and RNA sequencing data for these cell lines. To develop a model for each drug in the database, we used Elastic-Net regression, a regularized regression method that is a linear combination of the LASSO and Ridge methods. The Elastic-Net regression model is a penalized approach that produces biased coefficient estimates with a resulting decrease in variance, which can lead to an improvement in predictions compared to what can be achieved with a non-penalized regression model. This method also allows for feature selection, with coefficients of non-predictive features falling to zero or near-zero. To determine the optimized trade-off in bias and variance, cross validation is utilized to tune the two hyper-parameters of this model: the strength of the penalization (λ) and the proportion of LASSO versus Ridge penalty (α). An Elastic-Net model⁵⁵ was trained for all drugs in GDSC using the R caret wrapper for the GLMNET package, using the default parameters. Values for α and λ, were selected using 10-fold cross validation. The reported Z-score of the half-maximal inhibitory concentration (IC₅₀)¹² of each drug experiment was used as the measure of response in our model. The final output model from the Elastic-Net training procedure is in the form of a standard linear model, and the intercept and coefficients of all models described below can be found in Tables 1A-1I. The predictions from these models represent the TARGETS scores. Of note, immunotherapies were not tested in the GDSC and are not represented in TARGETS because these depend on the interaction between the tumor and host immune system, which was not modeled in the GDSC cell line experiments.

### In Vitro Validation

Independent validation of cell line drug response predictions was performed in the CCLE dataset¹⁶. RNA and DNA sequencing data were downloaded from the CCLE website (portals.broadinstitute.org/ccle). Gene expression and mutation data were normalized and represented the same way as the GDSC, detailed above. Predictions were made using the locked models previously trained in GDSC. Eighteen previously trained drug-models from the GDSC had matching drug response data available in the CCLE. In the CCLE dataset, 55% of all the IC₅₀ values were 8 μM (the maximal tested concentration). Thus, we utilized the AUC instead, which provides drug response information even if the IC₅₀ was not reached. Since higher AUC is associated with lower IC₅₀, we then compared the negative AUC determined from CCLE samples and compared to the GDSC predicted IC₅₀ to determine the correlation between our two predictions. A Pearson's correlation coefficient was determined for all 18 comparisons and the Benjamini Hochberg False Discovery Rate (FDR) was reported for each comparison to control for multiple testing.

### In Vivo Validation

TARGETS performance was evaluated in two clinical datasets: TCGA and the Stand Up to Cancer/Prostate Cancer Foundation WCDT. The TCGA processed sequencing and clinical data were downloaded using the UCSC Xena browser (xena.ucsc.edu)⁶³. The WCDT dataset contains 100 patients with mCRPC with both DNA and RNA sequencing²⁶, with Whole Genome Sequencing and RNA-seq data available at dbGAP (phs001648.v2.p1). We paired these data with previously unreported treatment response data to validate the ability of TARGETS to predict treatment response in this unique clinical cohort. Gene expression and mutation data were normalized and represented in the same manner as for both in vitro datasets. Predictions were made with the GDSC-trained and locked models without modification. Comparisons of predicted Z-score IC₅₀ between groups were performed using a T-test. Of note, the ARSI model as derived in GDSC was based on Bicalutamide, the only ARSI included in the training dataset. The ARSIs used in the WCDT were Enzalutamide and Abiraterone.

### Identifying Novel Biomarker-Drug Pairs

We utilized the TARGETS predictions detailed above to globally identify mutations associated with predicted drug sensitivity in TCGA. A linear model was used for this step, and tumor site was also included to identify pan-cancer biomarker-drug pairs. This approach identified mutations that were associated with drug sensitivity, independent of the disease site. Only named drugs further along in the regulatory process⁶⁴ and mutations with a >5% frequency across all cancers were included. The t-statistic of the mutation in the linear model was used to rank the mutation-drug pairs, and the top 1% were selected for further investigation.

### Data Availability

The data that support the findings of this study are available through the following locations. The Genomics of Drug Sensitivity in Cancer (GDSC) data were downloaded from the GDSC website (World Wide Web at cancerrxgene.org). The Cancer Cell Line Encyclopedia (CCLE) dataset were downloaded from the CCLE website (portals.broadinstitute.org/ccle). The TCGA processed sequencing and clinical data were downloaded using the UCSC Xena browser (xena.ucsc.edu). The WCDT dataset with Whole Genome Sequencing and RNA-seq data is available at dbGAP (phs001648.v2.p1).

### Code Availability

The TARGETS models are represented by linear models, and the coefficients are available in Tables 1A-1I which allow for generation of the TARGETS scores. All analysis was performed using R 4.0.3. Models were generated using R “caret” package with the following parameters: method=glmnet, trControl=trainControl(method=“repeatedCV”, number=10, repeats=10).

### Results

### Training Models on the GDSC Database

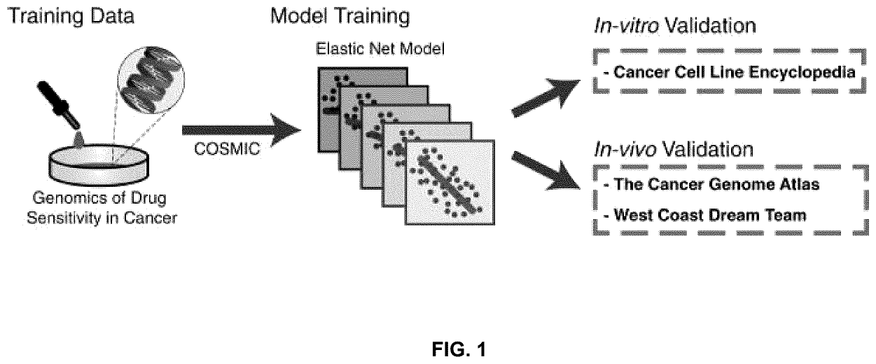

Our training cohort was the publicly available Genomics of Drug Sensitivity in Cancer (GDSC) database¹²⁻¹⁴ (FIG. 1). To reduce the noise in the data, we included only genes identified by the COSMIC Cancer Gene Census¹⁵. This critical step allowed us to leverage the extensive knowledge on cancer genomics to improve the signal-to-noise ratio and prediction accuracy. Elastic-Net regression models were then trained using the RNA expression and DNA mutation data on only the COSMIC genes for all treatments in the GDSC. The TARGETS models were locked and used for all subsequent predictions without modification.

Concordance with CCLE Drug Sensitivity

We next examined if the TARGETS predictions could successfully predict cell line drug response in an independent dataset from the Cancer Cell Line Encyclopedia (CCLE)¹⁶. Eighteen drugs were present in both CCLE and GDSC, allowing us to independently validate the performance of those 18 TARGETS models in CCLE. We compared the TARGETS predictions with the drug sensitivities in CCLE and found that 18 out of 18 were significantly correlated after adjusting for multiple testing (FDRs<0.05, Table 3). Validation of all models in an independent cell line drug response cohort provides additional experimental evidence supporting the TARGETS approach.

TABLE 3
TARGETS predictions correlate with CCLE drug sensitivity.

| Drug | Corr. Coef. | P-value | FDR |
|---|---|---|---|
| Nilotinib | 0.6 [0.66, 0.53] | 5.95E−38 | 2.14E−37 |
| Tanespimycin | 0.25 [0.33, 0.16] | 8.95E−08 | 1.07E−07 |
| PHA-665752 | 0.24 [0.33, 0.15] | 1.45E−07 | 1.63E−07 |
| Lapatinib | 0.49 [0.56, 0.42] | 4.06E−29 | 9.13E−29 |
| Nutlin-3a (−) | 0.26 [0.35, 0.18] | 1.11E−08 | 1.43E−08 |
| Saracatinib | 0.22 [0.31, 0.13] | 1.38E−06 | 1.46E−06 |
| Crizotinib | 0.45 [0.52, 0.37] | 6.06E−24 | 1.09E−23 |
| Panobinostat | 0.61 [0.67, 0.55] | 1.83E−48 | 3.29E−47 |
| Sorafenib | 0.42 [0.49, 0.34] | 1.45E−20 | 2.18E−20 |
| Irinotecan | 0.65 [0.71, 0.57] | 1.11E−35 | 3.33E−35 |
| Topotecan | 0.58 [0.64, 0.52] | 3.97E−43 | 3.57E−42 |
| PD0325901 | 0.57 [0.63, 0.51] | 1.27E−41 | 7.63E−41 |
| Palbociclib | 0.23 [0.32, 0.14] | 3.45E−06 | 3.45E−06 |
| Paclitaxel | 0.51 [0.57, 0.44] | 2.97E−31 | 7.64E−31 |
| Selumetinib | 0.48 [0.55, 0.4] | 1.51E−27 | 3.03E−27 |
| PLX-4720 | 0.57 [0.63, 0.51] | 7.66E−41 | 3.45E−40 |
| NVP-TAE684 | 0.34 [0.42, 0.26] | 6.76E−14 | 9.36E−14 |
| Erlotinib | 0.45 [0.52, 0.37] | 1.13E−23 | 1.84E−23 |

Pearson's correlation between TARGETS predictions in CCLE and drug sensitivity as measured by the negative AUC.
FDR Benjamini Hochberg corrected false discovery rate.

Concordance with Known Biomarker-Drug Combinations in the TCGA

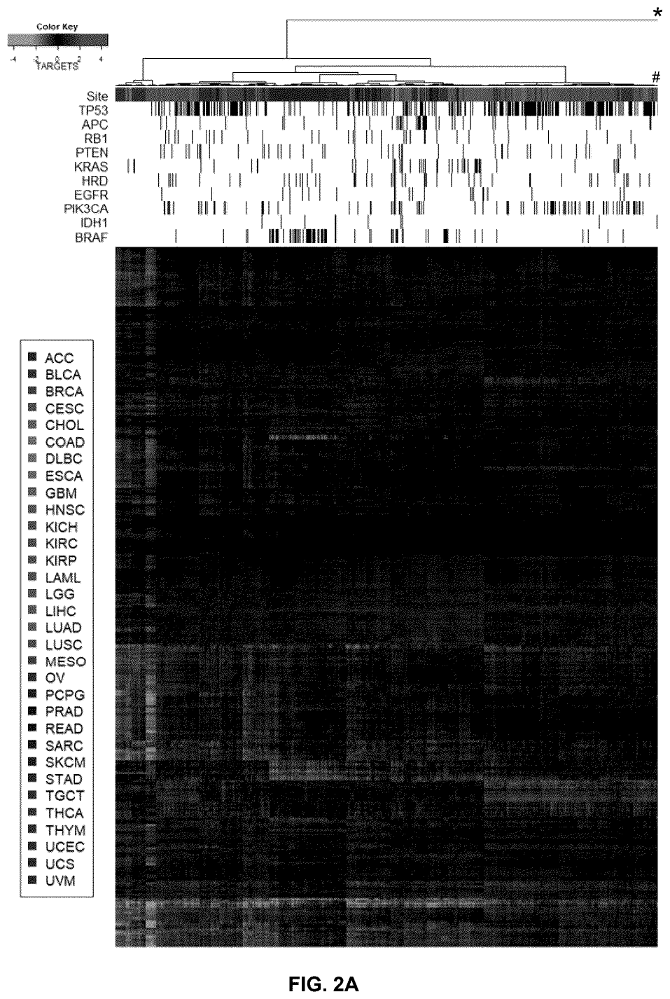

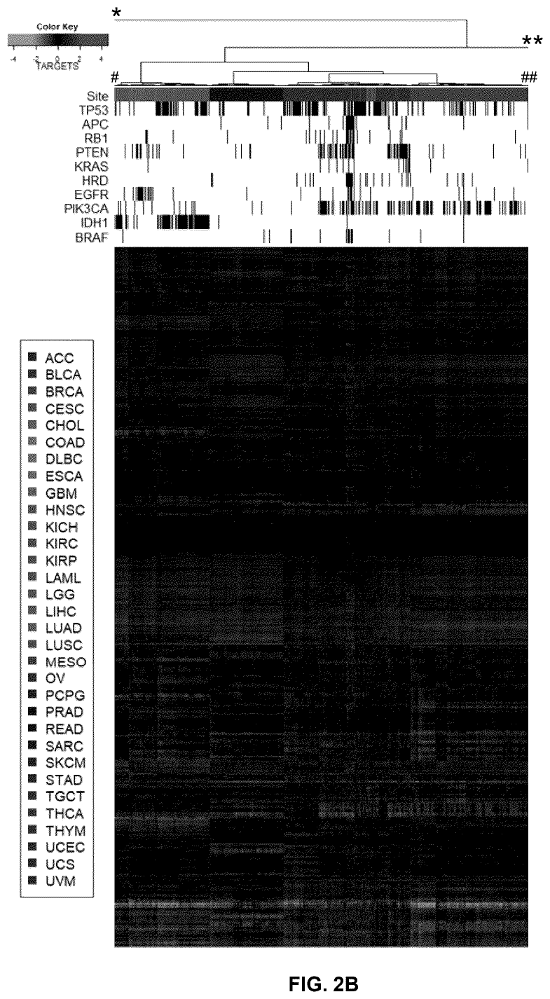

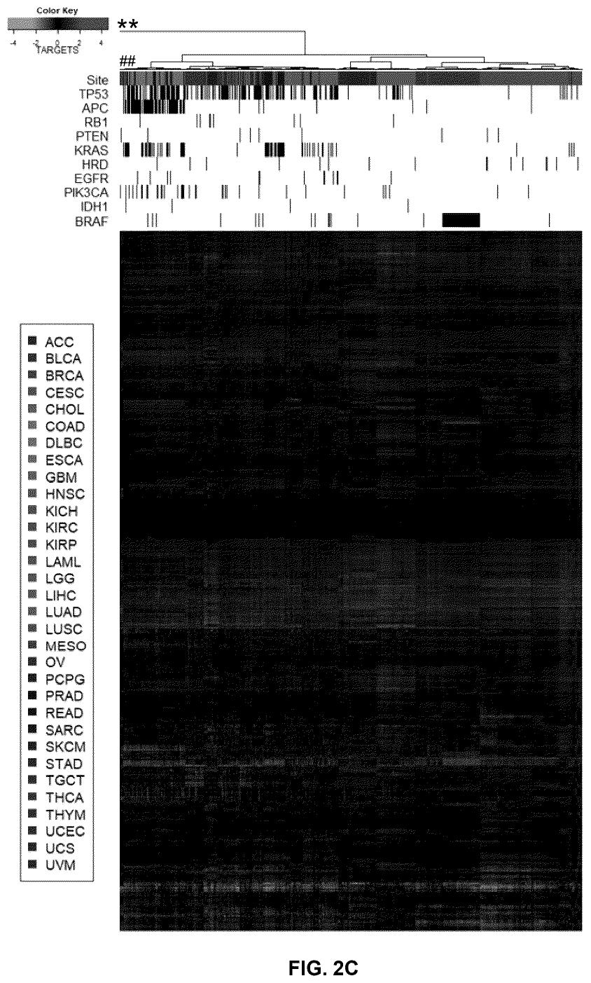

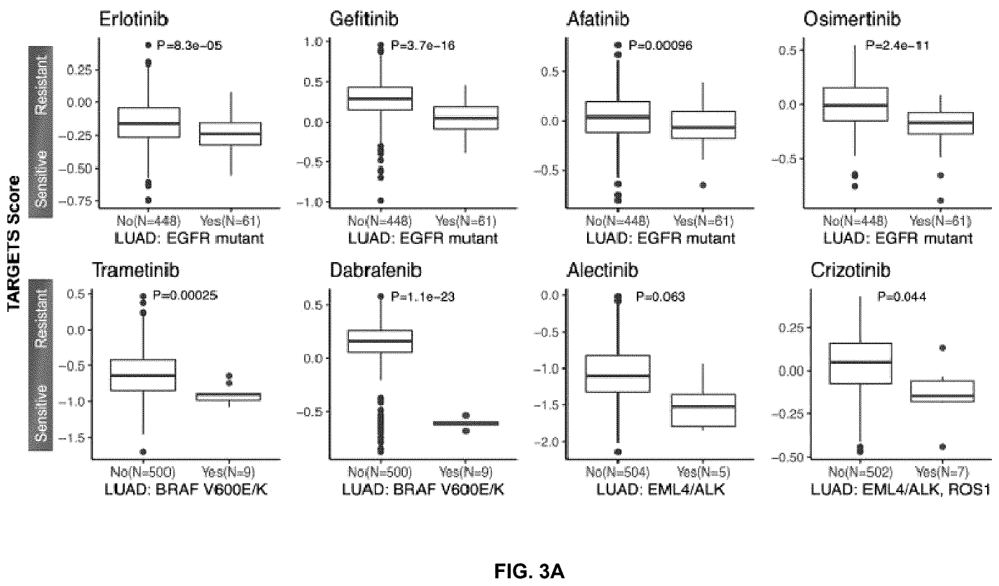

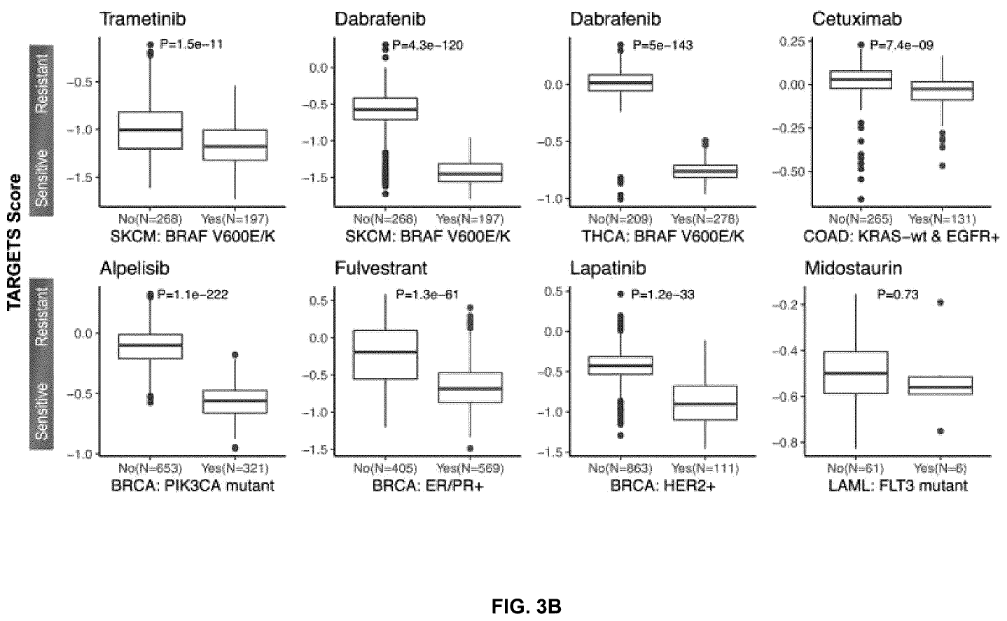

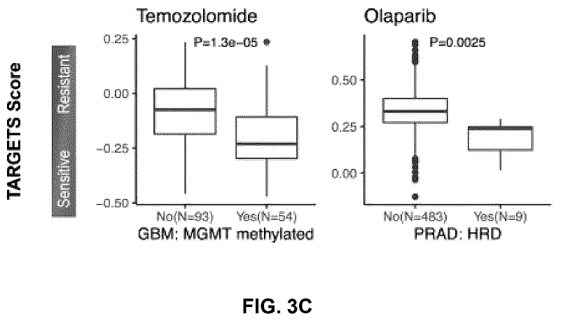

Data on 9430 patients from 32 cancer types from The Cancer Genome Atlas (TCGA) 17 was used to compare TARGETS with known biomarker-drug combinations. The distribution of predicted sensitivities varies widely across tumors and drugs. When we plotted the TARGETS predictions for all drugs across all tumor types, we observed that samples with the same tumor types tended to cluster together, as well as certain DNA alterations which tend to be highly enriched in certain tumor types (FIGS. 2A-2C). This is consistent with the evidence that many anti-cancer drugs tend to work better in specific tumor types, an assumption underlying current clinical practice. However, there is also a minority of samples that appear to be dissimilar to their tissue-of-origin and cluster better with other tumor types, highlighting the limitation of tumor-type-driven treatment decisions and the potential benefit of a molecularly driven approach. Predictions of drug sensitivity using TARGETS were made for all drugs and samples. We next tested our TARGETS predictions against all FDA-approved somatic biomarker indications (Table 2). For all biomarker-drug combinations tested, differences in drug sensitivity as predicted by TARGETS were in line with what was expected based on the indication (FIGS. 3A-3C). EGFR mutated lung adenocarcinomas were predicted to be more sensitive to Erlotinib, Gefitinib, Afatinib and Osimertinib (all with p<0.0001). BRAF V600E/K mutated lung adenocarcinoma and cutaneous melanoma both were predicted to be more sensitive to Trametinib and Dabrafenib (all with p<0.001). BRAF V600E/K mutant thyroid cancer was also predicted to be more sensitive to Dabrafenib (p<0.0001). EML4/ALK fusion-positive lung adenocarcinoma was predicted to be borderline more sensitive to Alectinib (p=0.0632) and EML4/ALK or ROS1 fusion-positive lung adenocarcinoma was predicted to be more sensitive to Crizotinib (p=0.044). KRAS wild-type colon cancer with EGFR expression greater than the median was predicted to be more resistant to Cetuximab (p<0.0001). PIK3CA mutated breast tumors were predicted to be more sensitive to Alpelisib. ER/PR positive breast cancer by histologic assessment was predicted to be more sensitive to Fulvestrant (an ER degrader, p<0.0001) and HER2 positive breast cancer by histologic assessment was predicted to be more sensitive to Lapatinib (p<0.0001). Midostaurin was not predicted to be significantly more sensitive in FLT3 mutant AML. However, the complete response rate even in FLT3 wild-type AML treated with Midostaurin can be up to 74%^{18,19}. In addition to these FDA-approved indications, we tested other clinically used biomarker-drug combinations. In GBM, the benefit of Temozolomide is more pronounced in MGMT promoter methylated tumors²⁰⁻²³, and we found MGMT-methylated glioblastoma was predicted to be more sensitive to Temozolomide (p<0.0001). PARP inhibitors, such as Olaparib, are now indicated for both HRD and non-HRD ovarian cancers, and we also did not find a significant difference in sensitivity to Olaparib between HRD and non-HRD ovarian cancers²⁴. However, in prostate cancer, HRD tumors were predicted to be more sensitive to Olaparib (p=0.0025), consistent with recent data from the phase III PROfound trial²⁵. These data therefore provide independent evidence that TARGETS predictions are concordant with FDA-approved biomarker indications.

### Predicting ARSI Response in Metastatic Prostate Cancer

Metastatic castration-resistant prostate cancer (mCRPC) is a common lethal cancer type not represented in the TCGA, and is commonly treated with ARSIs such as Enzalutamide or Abiraterone. This cancer type represents an opportunity to clinically validate our approach in an independent patient cohort. We utilized metastatic biopsy RNA and DNA sequencing data as well as ARSI response data on 100 patients from the Stand Up to Cancer/Prostate Cancer Foundation West Coast Prostate Cancer Dream Team (WCDT) cohort²⁶ to evaluate whether TARGETS could predict which patients may benefit from ARSI therapy. 50% PSA response is a common cutoff used in randomized trials in metastatic prostate cancer²⁷⁻³¹, and we used this as our primary clinical endpoint. We found that among patients receiving ARSIs as the next-line therapy after their biopsy, responders (defined as those who had 51-100% PSA response) were predicted to be more sensitive to ARSIs compared to the non-responders (0-50% PSA response) (FIG. 4A; p=0.0381). There was no difference in the predicted sensitivity to ARSIs of responders and non-responders who received other drugs (p=0.2143), providing a control that shows the model is specific in identifying patients who will respond to ARSIs rather than just identifying those who will have a good response to treatment in general. In a logistic regression model predicting PSA response, the interaction between ARSI treatment and TARGETS score was statistically significant (p=0.0252; FIG. 4B) indicating that TARGETS is a bona fide predictive biomarker for response to ARSIs³²⁻³⁶.

Exploratory Identification of Potential Therapeutic Strategies with TARGETS

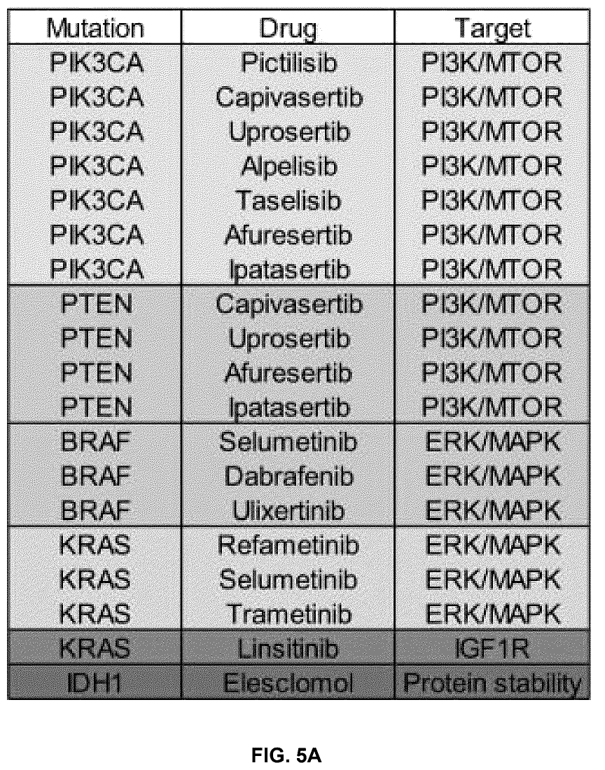

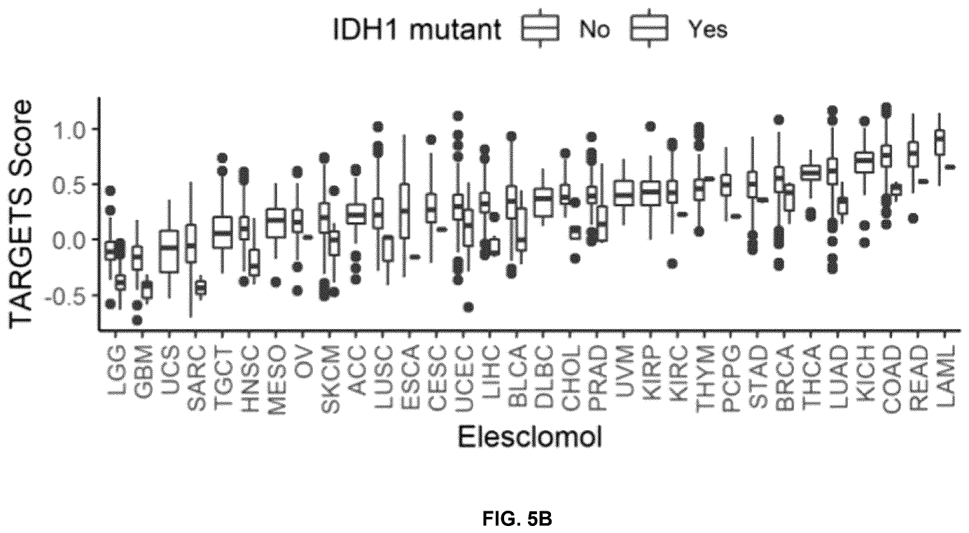

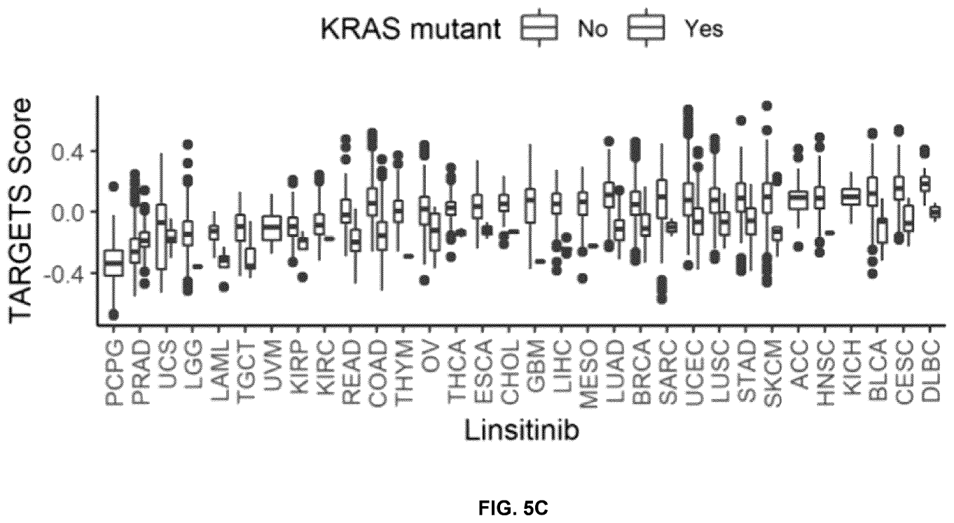

While mutations may occur randomly, those that provide a growth advantage are selected for in cancer. Frequent mutation of a gene may signal a tumor's dependence on that gene or pathway and therefore represents a potential therapeutic target. We hypothesized that examining specific mutations associated with TARGETS in clinical samples could identify known and novel therapeutic strategies. To this end, we identified the mutations most strongly correlated with TARGETS predictions in TCGA. The top 1% of putative mutation—drug sensitivity combinations are shown in FIG. 5A. Out of these 19 pairs, 17 were associations that would be reasonably expected given their mechanism of action (e.g., PIK3CA/PTEN mutations and PI3K/MTOR inhibitors, BRAF/KRAS mutations, and ERK/MAPK inhibitors). Overall, tumors with PIK3CA and PTEN mutations were predicted to be more sensitive to drugs that target the PI3K/MTOR pathway which is downstream of those genes. Tumors with KRAS and BRAF mutations were predicted to be more sensitive to drugs that target the ERK/MAPK pathway which is downstream of RAS/RAF signaling. In addition, Linsitinib, an IGF1R inhibitor, was predicted to be more effective in KRAS mutant tumors (FIG. 5B), consistent with experimental data in NSCLC³⁷. The final drug on the list, Elesclomol, was predicted to be more effective in IDH1 mutant tumors, especially gliomas (FIG. 5C), an association not previously reported in the literature. There were no IDH1 mutant LGG or GBM cell lines included in the GDSC, but TARGETS was nonetheless able to identify improved predicted Temozolomide response in MGMT methylated GBM patients (FIGS. 3A-3B). These predictions represent hypothesis-generating extrapolations that go beyond the original training data, which can be used to identify potential novel therapeutic strategies.

### DISCUSSION

Personalized genomic medicine has changed the paradigm of cancer treatment. Next-generation genomic sequencing has shifted treatment decisions from using radiologic and histologic data alone, to an approach that incorporates individualized molecular features. In this study, we set out to develop TARGETS, a pan-cancer, platform-independent model for predicting sensitivity to therapy based on RNA expression and DNA mutation profiles. TARGETS was then validated across three datasets: the in-vitro CCLE and in vivo TCGA and WCDT datasets. Our predicted results were concordant for all 18 drugs that were common between the CCLE and GDSC, and TARGETS had consistent predictions with known biomarker-drug indications across the TCGA. Furthermore, we independently validated TARGETS as a predictive biomarker for ARSI response in mCRPC in the WCDT cohort. Finally, we evaluated TARGETS use as a tool for hypothesis generation in identifying new drug indications.

Many attempts have been made to develop in vitro pharmacogenomic response signatures based on the publicly available GDSC, CCLE, and TCGA datasets^{14,16,38-47}. TARGETS demonstrates a stronger level of concordance across all known biomarker-drug indications in clinical samples than has been described in previously published studies⁴⁸. A few studies have also trained RNA-based signatures that were prognostic in clinical cohorts treated with specific agents⁴⁹⁻⁵¹. However, these studies have not necessarily identified predictive biomarkers, which are biomarkers that predict response only to a particular treatment, thus requiring validation data that includes un-treated patients³²⁻³⁶. This distinction is particularly important with regards to non-targeted therapies, such as traditional cytotoxic chemotherapies, which have been the focus of most of these prior studies. When no un-treated patient data exists, a signature for “response” may simply be measuring the overall aggressiveness of a tumor (e.g., prognosis), instead of providing truly predictive information specific to that agent. Statistical interaction testing, as we demonstrate, is required to identify truly predictive biomarkers³²⁻³⁶.

The primary challenge in assessing the performance of TARGETS is locating suitable clinical validation datasets with both multi-omics and treatment response data. There are in vitro pharmacogenomic databases such as the CCLE in which we were able to perform validation. The CCLE is similar to the GDSC, including many shared cell lines as both were designed to be comprehensive catalogues of cancer cell lines. However, the two cohorts were distinct efforts in time and space, and there were significant differences in culture conditions, gene expression profiling, drug screen procedures, and many other major and minor factors, to the extent that significant discordance between the two datasets has been reported^{52,53}. The validation of 100% of TARGETS predictions in CCLE despite these differences provides strong supporting evidence for the approach. Ideally, clinical validation would be performed for every drug in every disease site. However, there is a lack of clinical cohorts with both DNA and RNA sequencing and detailed response data from both treated and untreated patients. Datasets such as the TCGA have the former but not the latter. Furthermore, systemic therapies are primarily used in the later stages of the disease, but obtaining invasive metastatic biopsies for molecular profiling is not routine. The WCDT is a unique cohort with both comprehensive molecular profiling and ARSI drug response data making it the ideal clinical dataset in which to validate TARGETS. The rarity of such clinical datasets highlights the need for DNA and RNA profiling in larger prospective studies with detailed treatment and outcomes data.

We believe the model development strategy presented herein has yielded improved generalizability and interpretability. First, our approach is unique in that we use only genes known to be strongly associated with cancer from the literature′. While it initially seems counter-intuitive that removing information from the vast majority of genes would be beneficial, a genome-wide approach suffers from a great deal of noise. Not only are many genes not important to treatment response or resistance, but cell lines in particular acquire many passenger mutations over time. Therefore, by focusing on a small set of cancer-associated genes, changes in gene expression or the presence of mutations are more likely to be driving a biological function. Second, integration of both DNA and RNA information into our models can provide information on tumors driven by specific gene expression patterns (e.g., receptors in breast cancer) as well as specific DNA alterations (e.g., EGFR mutations in lung cancer)^{46,54}. Finally, we chose to utilize Elastic-Net regression⁵⁵, because this regularized approach is less prone to over-fitting⁵⁶ and thus would better handle the biological and technical differences between the in-vitro training data and the clinical datasets.

TARGETS may also be able to identify new therapeutic strategies. Interestingly, our results show that IDH1 mutations are the second most highly weighted feature in the model for Elesclomol, and that they are highly associated with predicted Elesclomol sensitivity. Elesclomol is a copper chelator that has been found to interact with the electron transport chain in mitochondria to generate high levels of reactive oxygen species (ROS)⁵⁷. IDH1 is well known for its role in the NADPH-dependent catalyzation of isocitrate to a-ketoglutarate (aKG), with IDH1 mutations leading to NADPH-dependent reduction of aKG to D-2-hydroxyglutarate (D2HG)⁵⁸. While D2HG has many downstream effects that contribute to tumorigenesis in IDH mutant tumors⁵⁹, this increased utilization of NADPH impacts the cell's ability to form a sufficient response to increased production of ROS. This mechanism could in part explain why IDH1-mutant glioma patients have better prognosis⁶⁰ and would mechanistically support our prediction of increased sensitivity to Elesclomol in IDH1-mutant tumors. To our knowledge, this association has not been previously documented in the literature and thus warrants further investigation to evaluate its use in IDH1-mutant tumors, particularly gliomas, which were predicted to have the greatest sensitivity to this agent with or without IDH1 mutation.

In conclusion, our study describes a pan-cancer, multi-omics approach for the identification of predictive biomarkers across tumor types. Many drugs demonstrate some efficacy in a minority of patients but lack sufficient clinical benefit in unselected populations to warrant FDA approval or clinical use. To date, we lack a unified global approach for identifying the patients most likely to benefit from specific therapies. TARGETS is platform-independent, and thus can be applied to a wide range of current and future datasets. RNA-seq should be normalized as described, and any DNA variant-calling pipeline can be used. There will of course be technical variation across different datasets. However, elastic-net regression is particularly well suited to handle some degree of noise, and our validation is on a variety of different platforms. TARGETS could be used in future clinical trials to select only patients most likely to benefit from the trial agent for inclusion, thus maximizing the chances of success.

### REFERENCES

1. Bleeker F E, Bardelli A. Genomic landscapes of cancers: prospects for targeted therapies. Pharmacogenomics. 2007; 8:1629-1633.
2. Druker B J. Perspectives on the development of a molecularly targeted agent. Cancer Cell. 2002; 1:31-36.
3. Cobleigh M A, et al. Multinational study of the efficacy and safety of humanized anti-HER2 monoclonal antibody in women who have HER2-overexpressing metastatic breast cancer that has progressed after chemotherapy for metastatic disease. J. Clin. Oncol. 1999; 17:2639-2639.
4. Russo, A. et al. Heterogeneous responses to epidermal growth factor receptor (EGFR) tyrosine kinase inhibitors (TKIs) in patients with uncommon EGFR mutations: new insights and future perspectives in this complex clinical scenario. Int. J. Mol. Sci.10.3390/ijms20061431 (2019).
5. Mitsudomi T, et al. Gefitinib versus cisplatin plus docetaxel in patients with non-small-cell lung cancer harbouring mutations of the epidermal growth factor receptor (WJTOG3405): an open label, randomised phase 3 trial. Lancet Oncol. 2010; 11:121-128.
6. Han J Y, et al. First-SIGNAL: first-line single-agent iressa versus gemcitabine and cisplatin trial in never-smokers with adenocarcinoma of the lung. J. Clin. Oncol. 2012; 30:1122-1128.
7. Zhou C, et al. Final overall survival results from a randomised, phase III study of erlotinib versus chemotherapy as first-line treatment of EGFR mutation-positive advanced non-small-cell lung cancer (OPTIMAL, CTONG-0802) Ann. Oncol. 2015; 26:1877-1883.
8. Wu Y L, et al. First-line erlotinib versus gemcitabine/cisplatin in patients with advanced EGFR mutation-positive non-small-cell lung cancer: analyses from the phase III, randomized, open-label, ENSURE study. Ann. Oncol. 2015; 26:1883-1889.
9. Rosell R, et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol. 2012; 13:239-246.
10. Paz-Ares L, et al. Afatinib versus gefitinib in patients with EGFR mutation-positive advanced non-small-cell lung cancer: overall survival data from the phase Iib LUX-Lung 7 trial. Ann. Oncol. 2017; 28:270-277.
11. Soria J C, et al. Osimertinib in untreated EGFR-mutated advanced non-small-cell lung cancer. N. Engl. J. Med. 2018; 378:113-125.
12. Yang W, et al. Genomics of drug sensitivity in cancer (GDSC): a resource for therapeutic biomarker discovery in cancer cells. Nucleic Acids Res. 2013;41:D955-D961.
13. Garnett, M. J. et al. Systematic identification of genomic markers of drug sensitivity in cancer cells. 483,570-575 (2012).
14. Iorio F, et al. A landscape of pharmacogenomic interactions in cancer. Cell. 2016; 166:740-754.
15. Tate J G, et al. COSMIC: the catalogue of somatic mutations in cancer. Nucleic Acids Res. 2019;47:D941-D947.
16. Barretina J, et al. The Cancer Cell Line Encyclopedia enables predictive modelling of anticancer drug sensitivity. Nature. 2012; 483:603-607.
17. Weinstein J N, et al. The Cancer Genome Atlas Pan-Cancer analysis project. Nat. Genet. 2013; 45:1113-1120.
18. Stone R M, et al. Phase D3 study of the FLT3 kinase inhibitor midostaurin with chemotherapy in younger newly diagnosed adult patients with acute myeloid leukemia. Leukemia. 2012; 26:2061-2068.
19. Stone R M, et al. Midostaurin plus chemotherapy for acute myeloid leukemia with a FLT3 mutation. N. Engl. J. Med. 2017; 377:454-464.
20. Hegi M E, et al. MGMT gene silencing and benefit from temozolomide in glioblastoma.

N. Engl. J. Med. 2005; 352:997-1003.
21. Malmstrom A, et al. Temozolomide versus standard 6-week radiotherapy versus hypofractionated radiotherapy in patients older than 60 years with glioblastoma: the Nordic randomised, phase 3 trial. Lancet Oncol. 2012; 13:916-926.
22. Perry J R, et al. Short-course radiation plus temozolomide in elderly patients with glioblastoma. N. Engl. J. Med. 2017; 376:1027-1037.
23. Wick W, et al. Temozolomide chemotherapy alone versus radiotherapy alone for malignant astrocytoma in the elderly: the NOA-08 randomised, phase 3 trial. Lancet Oncol. 2012; 13:707-715.
24. González-Martín A, et al. Niraparib in patients with newly diagnosed advanced ovarian cancer. N. Engl. J. Med. 2019; 381:2391-2402.
25. Sandhu S K, et al. PROfound: Phase III study of olaparib versus enzalutamide or abiraterone for metastatic castration-resistant prostate cancer (mCRPC) with homologous recombination repair (HRR) gene alterations. Ann. Oncol. 2019;30:ix188-ix189.
26. Quigley D A, et al. Genomic hallmarks and structural variation in metastatic prostate cancer. Cell. 2018; 175:889.
27. de Bono J S, et al. Abiraterone and increased survival in metastatic prostate cancer. N. Engl. J. Med. 2011; 364:1995-2005.
28. Hussain M, et al. Enzalutamide in men with nonmetastatic, castration-resistant prostate cancer. N. Engl. J. Med. 2018; 378:2465-2474.
29. Scher H I, et al. Increased survival with enzalutamide in prostate cancer after chemotherapy. N. Engl. J. Med. 2012; 367:1187-1197.
30. Smith M R, et al. Apalutamide treatment and metastasis-free survival in prostate cancer. N. Engl. J. Med. 2018; 378:1408-1418.
31. Fizazi K, et al. Darolutamide in nonmetastatic, castration-resistant prostate cancer. N. Engl. J. Med. 2019; 380:1235-1246.
32. Zhao S G, et al. Associations of luminal and basal subtyping of prostate cancer with prognosis and response to androgen deprivation therapy. JAMA Oncol. 2017; 3:1663-1672.
33. Zhao, S. G. et al. Development and validation of a 24-gene predictor of response to postoperative radiotherapy in prostate cancer: a matched, retrospective analysis. Lancet Oncol. 10.1016/S1470-2045(16)30491-0 (2016).
34. Zhao S G, et al. The immune landscape of prostate cancer and nomination of PD-L2 as a potential therapeutic target. J. Natl Cancer Inst. 2019; 111:301-310.
35. Zhao S G, et al. Xenograft-based platform-independent gene signatures to predict response to alkylating chemotherapy, radiation, and combination therapy for glioblastoma. Neuro Oncol. 2019
36. Ballman K V. Biomarker: predictive or prognostic? J. Clin. Oncol. 2015; 33:3968-3971.
37. Molina-Arcas M, Hancock D C, Sheridan C, Kumar M S, Downward J. Coordinate direct input of both KRAS and IGF1 receptor to activation of PI3 kinase in KRAS-mutant lung cancer. Cancer Discov. 2013; 3:548-563.
38. Polano, M. et al. A pan-cancer approach to predict responsiveness to immune checkpoint inhibitors by machine learning. Cancers10.3390/cancers11101562 (2019).
39. Reinhold W C, et al. Using drug response data to identify molecular effectors, and molecular “omic” data to identify candidate drugs in cancer. Hum. Genet. 2015; 134:3-11. Doi: 10.1007/s00439-014-1482-9.
40. Wang X, Sun Z, Zimmermann M T, Bugrim A, Kocher J P. Predict drug sensitivity of cancer cells with pathway activity inference. BMC Med. Genomics. 2019; 12:15.
41. Dhruba S R, Rahman R, Matlock K, Ghosh S, Pal R. Application of transfer learning for cancer drug sensitivity prediction. BMC Bioinforma. 2018; 19:497.
42. Suphavilai C, Bertrand D, Nagarajan N. Predicting cancer drug response using a recommender system. Bioinformatics. 2018; 34:3907-3914.
43. Wang L, Li X, Zhang L, Gao Q. Improved anticancer drug response prediction in cell lines using matrix factorization with similarity regularization. BMC Cancer. 2017; 17:513.
44. Pleasance E, et al. Pan-cancer analysis of advanced patient tumors reveals interactions between therapy and genomic landscapes. Nat. Cancer. 2020; 1:452-468.
45. Sharifi-Noghabi, H., Peng, S., Zolotareva, O., Collins, C. C. & Ester, M. AITL: Adversarial Inductive Transfer Learning with input and output space adaptation for pharmacogenomics. bioRxiv, 2020.2001.2024.918953 (2020).
46. Sharifi-Noghabi H, Zolotareva 0, Collins C C, Ester M. MOLI: multi-omics late integration with deep neural networks for drug response prediction. Bioinformatics. 2019;35:i501-i509.
47. Yang J, Li A, Li Y, Guo X, Wang M. A novel approach for drug response prediction in cancer cell lines via network representation learning. Bioinformatics. 2019; 35:1527-1535.
48. Geeleher P, et al. Discovering novel pharmacogenomic biomarkers by imputing drug response in cancer patients from large genomics studies. Genome Res. 2017; 27:1743-1751.
49. Sakellaropoulos T, et al. A deep learning framework for predicting response to therapy in cancer. Cell Rep. 2019; 29:3367-3373.e3364.
50. Lu, T. P. et al. Developing a prognostic gene panel of epithelial ovarian cancer patients by a machine learning model. Cancers10.3390/cancers11020270 (2019).
51. Geeleher P, Cox N J, Huang R S. Clinical drug response can be predicted using baseline gene expression levels and in vitro drug sensitivity in cell lines. Genome Biol. 2014;15:R47. Doi: 10.1186/gb-2014-15-3-r47.
52. Haibe-Kains B, et al. Inconsistency in large pharmacogenomic studies. Nature. 2013; 504:389-393.
53. Safikhani Z, et al. Revisiting inconsistency in large pharmacogenomic studies. F1000Res. 2016; 5:2333.
54. Rodon J, et al. Genomic and transcriptomic profiling expands precision cancer medicine: the WINTHER trial. Nat. Med. 2019; 25:751-758.
55. Zou H, Hastie T. Regularization and variable selection via the elastic net. J. R. Stat. Soc. Ser. B (Stat. Methodol.) 2005; 67:301-320.
56. Rhys, H. I. Machine Learning with R, the tidyverse, and mlr. 1^{st} edn. (Manning Publications, 2020).
57. Blackman R K, et al. Mitochondrial electron transport is the cellular target of the oncology drug elesclomol. PloS ONE. 2012; 7:e29798.
58. Bergaggio, E. & Piva, R. Wild-type IDH enzymes as actionable targets for cancer therapy. Cancers10.3390/cancers11040563 (2019).
59. Tommasini-Ghelfi S, et al. Cancer-associated mutation and beyond: The emerging biology of isocitrate dehydrogenases in human disease. Sci. Adv. 2019;5:eaaw4543.
60. Kaminska, B., Czapski, B., Guzik, R., Krol, S. K. & Gielniewski, B. Consequences of IDH1/2 mutations in gliomas and an assessment of inhibitors targeting mutated IDH proteins. Molecules10.3390/molecu1es24050968 (2019).
61. Torres-Garcia, W. et al. PRADA: pipeline for RNA sequencing data analysis. 30, 2224-2226 (2014).
62. Spano J P, et al. Epidermal growth factor receptor signaling in colorectal cancer: preclinical data and therapeutic perspectives. Ann. Oncol. 2005; 16:189-194.
63. Goldman M J, et al. Visualizing and interpreting cancer genomics data via the Xena platform. Nat. Biotechnol. 2020; 38:675-678. Doi: 10.1038/s41587-020-0546-8.
64. Karet G B. How do drugs get named? AMA J. Ethics. 2019;21:E686-E696.
65. Rydzewski N R, Peterson E, Lang J M, Yu M, Laura Chang S, Sjostrom M, Bakhtiar H, Song G, Helzer K T, Bootsma M L, Chen W S, Shrestha R M, Zhang M, Quigley D A, Aggarwal R, Small E J, Wahl D R, Feng F Y, Zhao S G. Predicting cancer drug TARGETS—TreAtment Response Generalized Elastic-neT Signatures. NPJ Genom Med. 2021 Sep. 21; 6(1):76.

### EXEMPLARY EMDOBIMENTS

1. A method of predicting response of a patient afflicted with a disease to a treatment and, optionally, administering the treatment to the patient, the method comprising:
determining a gene expression level for each of one or more first genes in a patient sample comprising pathological patient cells;
determining a mutation status for each of one or more second genes in the patient sample; and
determining a treatment-response score from the one or more gene expression levels and the one or more mutation statuses in a linear regression predictor model that includes a predictor intercept, a predictor gene-expression coefficient for each of the one or more first genes, and a predictor mutation-status coefficient for each of the one or more second genes, wherein the treatment-response score indicates a predicted response of the patient to the treatment.

2. The method of exemplary embodiment 1, comprising isolating the patient sample from the patient.

3. The method of any prior exemplary embodiment, wherein:
the determining the gene expression level for each of the one or more first genes comprises assaying the gene expression level of each of the one or more first genes in the patient sample; and
the determining the mutation status for each of the one or more second genes comprises assaying the mutation status of each of the one or more second genes in the patient sample.

4. The method of any prior exemplary embodiment, wherein the patient is a cancer patient, the treatment is a cancer treatment, and the patient sample comprises cancer cells.

5. The method of any prior exemplary embodiment, wherein the mutation status indicates presence or absence of a coding mutation.

6. The method of any prior exemplary embodiment, wherein the one or more first genes, the one or more second genes, the predictor intercept, the one or more predictor gene-expression coefficients, and the one or more predictor mutation-status coefficients are determined by a process comprising:
identifying one or more disease-associated genes that are associated with the disease;
determining treatment responses of training samples comprising pathological training cells subjected to the treatment;
determining a gene expression level and a mutation status for each disease-associated gene in each training sample;
modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.

7. The method of exemplary embodiment 6, wherein the one or more first genes comprise all the disease-associated genes having a non-zero training gene-expression coefficient.

8. The method of any one of exemplary embodiments 6-7, wherein the one or more second genes comprise all the disease-associated genes having a non-zero training mutation-status coefficient.

9. The method of any one of exemplary embodiments 6-8, wherein the linear regression training model is a penalized linear regression model.

10. The method of any one of exemplary embodiments 6-9, wherein the linear regression training model is an Elastic-Net regression model.

11. The method of any one of exemplary embodiments 6-10, wherein:
the determining the treatment responses of the training samples comprises assaying responses of the training samples to the treatment;
the determining the gene expression level for each disease-associated gene in each training sample comprises assaying the gene expression level for each disease-associated gene in each training sample; and
the determining the mutation status for each disease-associated gene in each training sample comprises assaying the mutation status for each disease-associated gene in each training sample.

12. The method of any one of exemplary embodiments 6-11, wherein the patient is a cancer patient, the treatment is a cancer treatment, the patient sample comprises cancer cells, the disease-associated genes are cancer-associated genes, and the training samples comprise cancer cells.

13. The method of any prior exemplary embodiment, wherein:
the treatment is a treatment with a drug listed in Tables 1A-1I;
the one or more first genes comprise any one or more genes listed in Tables 1A-1I that have a non-zero gene-expression coefficient for the drug;
the one or more second genes comprise any one or more genes listed in Tables 1A-1I that have a non-zero mutation-status coefficient for the drug;
the predictor intercept is an approximate of the intercept listed in Tables 1A-1I for the drug;
each predictor gene-expression coefficient is an approximate of the gene-expression coefficient for one of the one or more first genes listed in Tables 1A-1I for the drug; and
each predictor mutation-status coefficient is an approximate of the mutation-status coefficient for one of the one or more second genes listed in Tables 1A-1I for the drug.

14. The method of exemplary embodiment 13, wherein the one or more first genes comprise all the genes listed in Tables 1A-1I that have a non-zero coefficient for the drug.

15. The method of any one of exemplary embodiments 13-14, wherein the one or more second genes comprise all the genes listed in Tables 1A-1I having a non-zero coefficient for the drug.

16. The method of any one of exemplary embodiments 13-15, wherein the determining the treatment-response score comprises determining a treatment-response score for more than one drug listed in Tables 1A-1I using a different linear regression predictor model for each of the more than one drug.

17. The method of any prior exemplary embodiment, further comprising administering the treatment to the patient.

18. The method of any one of exemplary embodiments 1-16, further comprising administering the treatment to the patient if the treatment-response score is within a therapeutic range.

19. The method of any one of exemplary embodiments 17-18, wherein the administering ameliorates the disease.

20. A method of generating a linear regression predictor model capable of predicting response of a patient afflicted with a disease to a treatment, the linear regression predictor model comprising one or more first genes, one or more second genes, a predictor intercept, one or more predictor gene-expression coefficients, and one or more predictor mutation-status coefficients, the method comprising:
identifying one or more disease-associated genes that are associated with the disease;
determining treatment responses of training samples comprising pathological training cells subjected to the treatment;
determining a gene expression level and a mutation status for each disease-associated gene in each training sample;
modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.

21. The method of exemplary embodiment 20, wherein the one or more first genes comprise all the disease-associated genes having a non-zero training gene-expression coefficient.

22. The method of any one of exemplary embodiments 20-21, wherein the one or more second genes comprise all the disease-associated genes having a non-zero training mutation-status coefficient.

23. The method of any one of exemplary embodiments 20-22, wherein the linear regression training model is a penalized linear regression model.

24. The method of any one of exemplary embodiments 20-23, wherein the linear regression training model is an Elastic-Net regression model.

25. The method of any one of exemplary embodiments 20-24, wherein:
the determining the treatment responses of the training samples comprises assaying responses of the training samples to the treatment;
the determining the gene expression level for each disease-associated gene in each training sample comprises assaying the gene expression level for each disease-associated gene in each training sample; and
the determining the mutation status for each disease-associated gene in each training sample comprises assaying the mutation status for each disease-associated gene in each training sample.

26. The method of any one of exemplary embodiments 20-25, wherein the disease-associated genes are cancer-associated genes and the training samples comprise cancer cells.

What is claimed is:

1. A method of predicting response of a patient afflicted with a disease to a treatment and, optionally, administering the treatment to the patient, the method comprising:
determining a gene expression level for each of one or more first genes in a patient sample comprising pathological patient cells;
determining a mutation status for each of one or more second genes in the patient sample; and
determining a treatment-response score from the one or more gene expression levels and the one or more mutation statuses in a linear regression predictor model that includes a predictor intercept, a predictor gene-expression coefficient for each of the one or more first genes, and a predictor mutation-status coefficient for each of the one or more second genes, wherein the treatment-response score indicates a predicted response of the patient to the treatment.

2. The method of claim 1, comprising isolating the patient sample from the patient.

3. The method of claim 1, wherein:
the determining the gene expression level for each of the one or more first genes comprises assaying the gene expression level of each of the one or more first genes in the patient sample; and
the determining the mutation status for each of the one or more second genes comprises assaying the mutation status of each of the one or more second genes in the patient sample.

4. The method of claim 1, wherein the patient is a cancer patient, the treatment is a cancer treatment, and the patient sample comprises cancer cells.

5. The method of claim 1, wherein the mutation status indicates presence or absence of a coding mutation.

6. The method of claim 1, wherein the one or more first genes, the one or more second genes, the predictor intercept, the one or more predictor gene-expression coefficients, and the one or more predictor mutation-status coefficients are determined by a process comprising:
identifying one or more disease-associated genes that are associated with the disease;
determining treatment responses of training samples comprising pathological training cells subjected to the treatment;
determining a gene expression level and a mutation status for each disease-associated gene in each training sample;
modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.

7. The method of claim 6, wherein the one or more first genes comprise all the disease-associated genes having a non-zero training gene-expression coefficient.

8. The method of claim 6, wherein the one or more second genes comprise all the disease-associated genes having a non-zero training mutation-status coefficient.

9. The method of claim 6, wherein the linear regression training model is a penalized linear regression model.

10. The method of claim 6, wherein the linear regression training model is an Elastic-Net regression model.

11. The method of claim 6, wherein:
the determining the treatment responses of the training samples comprises assaying responses of the training samples to the treatment;
the determining the gene expression level for each disease-associated gene in each training sample comprises assaying the gene expression level for each disease-associated gene in each training sample; and
the determining the mutation status for each disease-associated gene in each training sample comprises assaying the mutation status for each disease-associated gene in each training sample.

12. The method of claim 6, wherein the patient is a cancer patient, the treatment is a cancer treatment, the patient sample comprises cancer cells, the disease-associated genes are cancer-associated genes, and the training samples comprise cancer cells.

13. The method of claim 1, wherein:
the treatment is a treatment with a drug listed in Tables 1A-1I;
the one or more first genes comprise any one or more genes listed in Tables 1A-1I that have a non-zero gene-expression coefficient for the drug;
the one or more second genes comprise any one or more genes listed in Tables 1A-1I that have a non-zero mutation-status coefficient for the drug;
the predictor intercept is an approximate of the intercept listed in Tables 1A-1I for the drug;
each predictor gene-expression coefficient is an approximate of the gene-expression coefficient for one of the one or more first genes listed in Tables 1A-1I for the drug; and
each predictor mutation-status coefficient is an approximate of the mutation-status coefficient for one of the one or more second genes listed in Tables 1A-1I for the drug.

14. The method of claim 13, wherein the one or more first genes comprise all the genes listed in Tables 1A-1I that have a non-zero coefficient for the drug.

15. The method of claim 13, wherein the one or more second genes comprise all the genes listed in Tables 1A-1I having a non-zero coefficient for the drug.

16. The method of claim 13, wherein the determining the treatment-response score comprises determining a treatment-response score for more than one drug listed in Tables 1A-1I using a different linear regression predictor model for each of the more than one drug.

17. The method of claim 1, further comprising administering the treatment to the patient.

18. The method of claim 1, further comprising administering the treatment to the patient if the treatment-response score is within a therapeutic range.

19. The method of claim 17, wherein the administering ameliorates the disease.

20. A method of generating a linear regression predictor model capable of predicting response of a patient afflicted with a disease to a treatment, the linear regression predictor model comprising one or more first genes, one or more second genes, a predictor intercept, one or more predictor gene-expression coefficients, and one or more predictor mutation-status coefficients, the method comprising:
identifying one or more disease-associated genes that are associated with the disease;
determining treatment responses of training samples comprising pathological training cells subjected to the treatment;
determining a gene expression level and a mutation status for each disease-associated gene in each training sample;
modeling in a linear regression training model the gene expression levels, the mutation statuses, and the treatment responses to thereby determine a training intercept, a training gene-expression coefficient for each disease-associated gene, and a training mutation-status coefficient for each disease-associated gene, wherein the predictor intercept is the training intercept, the one or more first genes comprise any one or more of the disease-associated genes having a non-zero training gene-expression coefficient, the one or more second genes comprise any one or more of the disease-associated genes having a non-zero training mutation-status coefficient, the one or more predictor gene-expression coefficients are the training gene-expression coefficients of the disease-associated genes constituting the one or more first genes, and the one or more predictor mutation-status coefficients are the training mutation-status coefficients of the disease-associated genes constituting the one or more second genes.
